SEQUENCE LISTING (14 sequences)

SEQ ID NO: 1 - DNA, 20 bp, Artificial Sequence
  Description of Artificial SequencePrimer
tagtcacaccggtgcggccg

SEQ ID NO: 2 - DNA, 19 bp, Artificial Sequence
  Description of Artificial SequencePrimer
gtagctcaccggtgacgtc

SEQ ID NO: 3 - DNA, 69 bp, Artificial Sequence
  Description of Artificial SequencePrimer
cagagaagactcttgggtttctgataggcactgactctctctgcctattacaagataaaaatatatcat

SEQ ID NO: 4 - DNA, 72 bp, Artificial Sequence
  Description of Artificial SequencePrimer
aaagaacctctgggtccaagggtagaccaccagcagcctaagggtgggacttgtttgttgcatttctagcca

SEQ ID NO: 5 - DNA, 23 bp, Artificial Sequence
  Description of Artificial SequencePrimer
acaagacaggtttaaggagacca

SEQ ID NO: 6 - DNA, 24 bp, Artificial Sequence
  Description of Artificial SequencePrimer
gtctgtttcccattctaaactgta

SEQ ID NO: 7 - DNA, 30 bp, Artificial Sequence
  Description of Artificial SequencePrimer
tacggctgtcatcacttagacctcaccctg

SEQ ID NO: 8 - DNA, 29 bp, Artificial Sequence
  Description of Artificial SequencePrimer
catgccatggtcaagaaatcacagccgaa

SEQ ID NO: 9 - DNA, 29 bp, Artificial Sequence
  Description of Artificial SequencePrimer
catgccatggcgtgatctgatccttcaac

SEQ ID NO: 10 - DNA, 32 bp, Artificial Sequence
  Description of Artificial SequencePrimer
tagtcacaccggtggttaactcgacatcttgg

SEQ ID NO: 11 - DNA, 31 bp, Artificial Sequence
  Description of Artificial SequencePrimer
gtagctcaccggtgcaatgtaacatcagaga

SEQ ID NO: 12 - DNA, 70 bp, Artificial Sequence
  Description of Artificial SequencePrimer
cagagaagactcttgggtttctgataggcactgactctctctgcctatttatcagggttattgtctcatg

SEQ ID NO: 13 - DNA, 71 bp, Artificial Sequence
  Description of Artificial SequencePrimer
caaagaacctctgggtccaagggtagaccaccagcagcctaagggtgggaggcgtgatctgatccttcaac

SEQ ID NO: 14 - DNA, 20 bp, Artificial Sequence
  Description of Artificial SequencePrimer
ggcagaatccagatgctcaa

(19) United States
(12) Patent Application Publication
(10) Pub. No.: US 2003/0162188 A1
(43) Pub. Date: Aug. 28, 2003

(54) Method for selection and agents useful for same

(76) Inventors: Panayiotis A. Ioannou, Victoria (AU); Duangporn Jamsai, Narkonpathom (TH); Mikhail Nefedov, Oakland, CA (US); Michael Orford, Nicosia (CY); Kumaran Narayanan, Selangor (MY)

Correspondence Address: Leopold Presser, SCULLY, SCOTT, MURPHY & PRESSER, 400 Garden City Plaza, Garden City, NY, 11530, US

(21) Appl. No.: 10/205,915

(22) Filed: Jul. 26, 2002

(30) Foreign Application Priority Data
Jul. 27, 2001 (AU) PR6665/01

(51) Int. Cl. C12Q 1/68; C12N 15/85

(52) U.S. Cl. 435/6; 435/455

(57) ABSTRACT

The present invention relates generally to a method for the selection of a modified nucleic acid molecule and to agents useful for same. More particularly, the present invention relates to a method for counterselecting nucleic acid molecules which have undergone targeted modification. The method of the present invention is useful, inter alia, for rapidly and accurately selecting correctly modified nucleic acid molecules, such as modified bacterial artificial chromosomes. The present invention is also directed to counterselection cassettes for use in the method of the invention and to modified nucleic acid molecules selected thereby.

### FIELD OF THE INVENTION

The present invention relates generally to a method for the selection of a modified nucleic acid molecule and to agents useful for same. More particularly, the present invention relates to a method for counterselecting nucleic acid molecules which have undergone targeted modification. The method of the present invention is useful, inter alia, for rapidly and accurately selecting correctly modified nucleic acid molecules, such as modified bacterial artificial chromosomes. The present invention is also directed to counterselection cassettes for use in the method of the invention and to modified nucleic acid molecules selected thereby.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

There is a need for a greater understanding of the structure and organisation of the genomes of higher eukaryotes. Genomic sequences are often much longer than coding sequences. Although the functions of most non-coding sequences are not known, it is clear that they may play a crucial role in regulation of gene expression, stability and evolution. In contrast to cDNA constructs driven by unrelated or viral promoters, gene transfer of large genomic fragments shows correct temporal- and tissue-specific gene expression (Jaenisch, R. (1988) Science 240:1468-1474; Brinster, R. L. et al. (1988) Proc. Natl. Acad. Sci. USA 85:836-840; Choi, T., Huang, M., Gorman, C. and Jaenisch, R. (1991) Mol Cell. Biol. 11:3070-3074; Lamb, B. T. et al. (1993) Nat. Genet. 5:22-30; Peterson, K. R., et al. (1996) Proc. Natl. Acad. Sci. USA 93:6605-6609; Porcu, S., et al. (1997) Blood 90:4602-4609; Raguz, S., et al. (1998) Dev. Biol. 201-26-42).

Bacterial Artificial Chromosomes (BACs) and PACs are used increasingly for long-range physical mapping, (Hubert, R. S. et al. (1997) Genomics 41:218-226; Nechiporuk, T. et al. (1997) Genomics 44:321-329) positional cloning of disease genes, (Wooster, R. et al. (1995) Nature 378:789-792) whole genome sequencing projects (Venter, J. C., Smith, H. L. and Hood, L. (1996) Nature 381:364-366; Marshall, E. and Pennisi, E. (1998) Science 280:994-995; Venter, J. C. et al. (1998) Science 280:1540-1542) and functional studies (Antoch, M. P., et al., (1997) Cell 89:655-667; Hejna, J. A. et al., (1998) Nucleic Acids Res. 26:1124-1125). High quality BAC/PAC genomic libraries are much easier to construct than YAC libraries, for example, because of greater cloning efficiency in bacteria. BACs/PACs are maintained at 1-2 copies per cell in a well-defined recombination-deficient E. coli strain, DH10B, where they exhibit high clonal stability over many generations. Although they carry inserts up to about 300 kb in size, they can be purified in large quantities for functional studies through conventional bacterial plasmid isolation methods. The genomic inserts in these clones are large enough to preserve the integrity of most human genetic loci and are thus ideal for functional studies.

The completion of the Human Genome Sequencing Project together with the creation of PAC and BAC genomic libraries have highlighted the need for functional studies using intact genomic loci and the use of such loci for the development of accurate animal models. The size of genomic inserts in PAC/BAC libraries (100-300 kb) is large enough to contain most genes as intact functional units (Shizuya, H., Birren, B., Kim, U. J., Mancino, V., Slepak, T., Tachiiri, Y. and Simon, M. (1992) Proc. Nal. Acad. Sci. 89:8794-8797; Ioannou, P. A., Amemiya, C. T., Garnes, J., Kroisel, P. M., Shizuya, H., Chen, C., Batzer, M. A. and de Jong, P. J. (1994) Nat. Genet. 6:84-89). The high redundancy of these libraries also allows the isolation of multiple, partially overlapping clones for each gene of interest (Reid, L. H., Davies, C., Cooper, P. R. Crider-Miller, S. J., Sait, S. N., Nowak, N. J., Evans, G., Stanbridge, E. J., de Jong, P., Shows, T. B., Weissman, b. e. and Higins, M. J. (1997) Genomics 43:366-375, Harada, K., Nishizaki, T., Maekawa, K., Kubota, H., Suzuki, M., Obno, T., Saski, K. and Soeda, E. (2000) Genomics 67:268-272). Expression studies on such ready-made and well-characterised clones with variable amounts of sequences at their 5′ and 3′ ends should greatly facilitate the localisation of distal regulatory elements. Such clones are also ideal for the identification of agents that are capable of modifying gene expression under physiologically relevant conditions. For such studies, a sensitive reporter gene needs to be fused in-frame with the coding sequence of the gene of interest, followed by the creation of a stable cell line using the modified clone. A variety of agents can then be assayed for their ability to modify the expression of the gene under physiologically relevant conditions in a high-throughput screening format.

There has recently been developed the GET Recombination system, an inducible homologous recombination system for the targeted modification of PACs and BACs in the recA deficient E. coli DH10B strain (Narayanan, K., Williamson, R., Zhang, Y., Stewart, A. F. and Ioannou, P. A. (1999) Gene Ther. 6:442-447; International Patent Publication No. WO 00/26396) in which most PAC and BAC libraries are made. The inhibition of the recBCD nuclease in DH10B cells by the tightly regulated expression of the gam gene, together with the simultaneous induction of the recE and recT genes, has allowed efficient homologous recombination between linear DNA fragments and BAC clones resident in such cells without compromising cell viability (Narayanan et al., 1999, supra). This system has been used to demonstrate the targeted insertion of the luciferase gene downstream of a common splicing mutation (Narayanan et al., 1999, supra) for the development of an assay for agents that may modify splicing specificity. Also, there has been demonstrated the insertion of the EGFP reporter gene at the start codon of each of the five globin genes in a 200 kb beta-globin BAC clone, with the simultaneous creation of targeted deletions ranging from a few base pairs to at least 44 kb in length (Orford, M., Nefedov, M., Vadolas, J., Zaibak, F., Williamson, R. and Ioannous, P. A. (2000) Nudleic Acids Res. 28.e84) and the expression of EGFP under the regulatory elements of the beta-globin locus.

The creation of accurate cell line and animal models for specific mutations using intact functional loci requires the insertion of such modifications in PAC/BAC clones without leaving behind any operational sequences. The GET Recombination system has previously been utilised in combination with a tetracycline counterselection cassette Nefedov, M., Williamson, R. and Ioannou, P. A. (2000) Nucleic Acids Res. 28:e79) to facilitate such modifications. In the first round of GET Recombination, the tetracycline resistance (tet^{R}) gene is inserted in the region of interest. In a second round of recombination the tet^{R} gene is knocked out by a PCR fragment carrying the desired modification. The identification of true recombinants is facilitated by plating the cells in the presence of chlorotetracycline/fusaric acid (cTc/FA), since expression of the tet^{R} gene in the presence of cTc/FA is toxic to the cells. However, the effectiveness of killing of cells expressing the tet^{R} gene in the presence of cTc/FA is not very high, while a non-specific toxicity of cTc/FA is also observed in cells lacking the tet^{R} gene, resulting in slow growth of bacterial colonies.

A number of other suicide genes have been used for the positive selection of recombinants in cloning experiments eg sacB (Pierce, J. C., Sauer, B. and Sternberg, N. (1992) Proc. Natl. Acad. Sci. 89:2056-2060), ccdB (Bernard, P. (1996) Biotechniques 21:320-323), the ompAR4 (Chan, R. Y., Palfree, R. G., congote, L. F. and Solomon, S. (1994) DNA Cell Biol. 13:311-319), the Hok gene (Bej, A. K., Perlin, M. H. and Atlas, R. M. (1988) Appl. Environ. Microbiol. 54:2472-2477), rcsB gene (Arakawa, Y., Wacharotayankun, R., Ohta, M., Shoji, K., Watahiki, M., Horii, T. and Kato, N. (1991) Gene 104:81-84), a modified EcoRI gene (Kuhn, I., Stephenson, F. H., Boyer, H. W. and Greene, P. J. (1986) Gene 42:253-263) and others.

Accordingly, there is an ongoing need to develop selection methods which accurately and rapidly select modified nucleic acid molecules, in particular modified PAC/BAC clones.

In work leading up to the present invention, the inventors have developed a counterselection system based on harnessing a range of means of introducing double-stranded nucleic acid breaks as counterselection markers for the introduction of modifications, such as targeted modifications, in nucleic acid molecules (for example in (PACs and BACs in E. Coli). In particular, the inventors have exemplified herein the use of restriction endonucleases such as the EcoRI and I-SceI endonuclease genes for their usefulness in selecting PAC and BAC clones which have undergone targeted modification using the GET Recombination system. The inventors have further demonstrated the high efficiency generation and selection of BAC clones incorporating deletions ranging from a few kilobases to about 150 kilobases in length. The capacity to generate and accurately select nucleic acid molecules, such as BACs, which have undergone precise modifications now facilitates, inter alia, the generation, accurate mapping and functional analysis of regulatory elements comprising the human chromosomes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide sequence information prepared using the programme PatentIn Version 3.1, presented herein after the bibliography. Each nucleotide sequence is identified in the sequence listing by the numeric indicator <210> followed by the sequence identifier (e.g. <210>1, <210>2, etc). The length, type of sequence (DNA, etc) and source of organism for each nucleotide sequence is indicated by information provided in the numeric indicator fields <211>, <212> and <213>, respectively. Nucleotide sequences referred to in the specification are defined in the information provided in numeric indicator field <400> followed by the sequence identifier (e.g. <400>1, <400>2, etc).

One aspect of the present invention is directed to a method for selecting a modified nucleic acid molecule or derivative or analogue thereof, said method comprising the steps of facilitating the interaction, in a host cell, of a counterselection marker with an unmodified nucleic acid molecule at a target modification region, which counterselection marker can facilitate the inducible degradation of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the degradation activity of said counterselection marker.

Another aspect of the present invention provides a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said endonuclease.

In another aspect there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible restriction endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said restriction endonuclease.

In still another aspect there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence encoding a restriction endonuclease or derivative, homologue, equivalent or mimetic of said restriction endonuclease and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing expression of said counterselection marker.

In yet another aspect there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence incorporating a restriction endonuclease cleavage site and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing cleavage at said restriction endonuclease cleavage site.

Still yet another aspect of the present invention provides a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence encoding EcoRI or derivative, homologue, equivalent or mimetic thereof and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing expression of said EcoRI.

In a further aspect there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence incorporating an I-SceI cleavage site and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing cleavage at said I-SceI cleavage site.

In another further aspect the present invention provides a method for selecting a modified BAC or derivative or analogue thereof said method comprising the steps of facilitating the interaction, in a DH10B cell, of a counterselection marker with an unmodified BAC at a target modification region, which counterselection marker is a nucleic acid sequence encoding EcoRI or derivative, homologue, equivalent or mimetic thereof and which can facilitate the inducible cleavage of said BAC, facilitating the modification of said BAC at said target modification region, which modification comprises the functional deletion of said counterselection marker from said BAC and selecting said modified BAC wherein said selection step comprises inducing expression of said EcoRI.

In yet another further aspect there is provided a method for selecting a modified BAC or derivative or analogue thereof said method comprising the steps of facilitating the interaction, in a DH10B cell, of a counterselection marker with an unmodified BAC at a target modification region, which counterselection marker is a nucleic acid sequence incorporating an I-SceI cleavage site and which can facilitate the inducible cleavage of said BAC, facilitating the modification of said BAC at said target modification region, which modification comprises the functional deletion of said counterselection marker from said BAC and selecting said modified BAC wherein said selection step comprises inducing cleavage at said I-SceI cleavage site.

In another further aspect the present invention provides a method for selecting a modified PAC or derivative or analogue thereof said method comprising the steps of facilitating the interaction, in a DH10B cell, of a counterselection marker with an unmodified PAC at a target modification region, which counterselection marker is a nucleic acid sequence encoding EcoRI or derivative, homologue, equivalent or mimetic thereof and which can facilitate the inducible cleavage of said PAC, facilitating the modification of said PAC at said target modification region, which modification comprises the functional deletion of said counterselection marker from said PAC and selecting said modified PAC wherein said selection step comprises inducing expression of said EcoRI.

In yet another further aspect there is provided a method for selecting a modified PAC or derivative or analogue thereof said method comprising the steps of facilitating the interaction, in a DH10B cell, of a counterselection marker with an unmodified PAC at a target modification region, which counterselection marker is a nucleic acid sequence incorporating an I-SceI cleavage site and which can facilitate the inducible cleavage of said PAC, facilitating the modification of said PAC at said target modification region, which modification comprises the functional deletion of said counterselection marker from said PAC and selecting said modified PAC wherein said selection step comprises inducing cleavage at said I-SceI cleavage site.

Still another aspect of the present invention contemplates modified nucleic acid molecules selected by the method of the present invention.

The present invention also extends to the use of said modified nucleic acid molecules in the treatment and/or diagnosis of patients. Methods of treatment include gene therapy regimens. The present invention also extends to methods of screening which utilise said modified nucleic acid molecules.

Another aspect of the present invention contemplates a pharmaceutical composition comprising modified nucleic acid molecules generated by the method of the present invention together with one or more pharmaceutically acceptable carriers and/or diluents.

Yet another aspect of the present invention is directed to a kit for facilitating selection of a modified nucleic acid molecule said kit comprising compartments adapted to contain any one or more of a counterselection marker, reagents useful for facilitating modification of a nucleic acid molecule and reagents useful for facilitating selection of said modified nucleic acid molecule. Further compartments may also be included, for example, to receive nucleic acid molecules such as any one or more of the nucleotide sequences which are the subject of modification, the host cells or the nucleic acid molecules required to facilitate recombination such as that induced by the GET Recombination system, the recE/rccT system of recombination or the bacteriophage lambda system.

### BRIEF DESCRIPTION OF THE DRAWINGS

FIG. 1 is a schematic representation of a map showing the main features of the pGETrec2 plasmid. The lacl^{q} gene with its own constitutive promoter was cloned from plasmid pGEX-1 into the SgrAI site of the pGETrec plasmid.

FIG. 2 is a diagramatic representation of the insertion of disease-causing mutations into a 200 kb globin BAC clone using the EcoRI/Kan^{R} cassette in two-stage GET Recombination. In the first stage the EcoRI/Kan^{R} cassette was inserted into intron 1 of the β-globin gene using homology arms corresponding to the targeted region of pEBAC/148β. Recombinant clones are identified on kanamycin plates, while expression of EcoRI is repressed by the constitutive expression of lacl^{q} from the pGETrcc2 plasmid. In the second stage of recombination, the EcoRI/Kan^{R} cassette was knocked out in two separate experiments by PCR fragments carrying either the Hb E mutation or a 4 bp deletion. Non-recombinant clones were eliminated by induction of the EcoRI gene by plating the cells on IPTG.

FIG. 3 is an image of (A) EcoRI restriction digestion of an unmodified 200 kb pEBAC/148β clone (lane 1) and two modified pEBAC/148β/Hb E clones (lanes 2, 3) carrying the Hb E G→A point mutation in exon 1 of the β-globin gene. The arrow points to the 5.5 kb EcoRI fragment from the β-globin gene on which the modification took place; (B) Southern blot analysis of the gel depicted in the panel (A) after hybridisation with a ³²P-labelled LUG probe; (C) Sequence analysis of a pEBAC/148β/Hb E clone, demonstrating the insertion of the Hb E G→A point mutation; (D) Sequence analysis of a pEBAC/148β/4 bp clone, demonstrating the insertion of the codons 41/42 (-TTCT) deletion mutation.

FIG. 4 is a graphical representation of polyacrylamide gel electrophoresis of total cell extract from DH10B (pEBAC/148β, pGETrec2) electrocompetent cells (induced for 40 min with 0.2% w/v L-arabinose) and incubated in SOC for different times. M, Protein standard marker (Low range, Bio-Rad); C, no induction with arabinose; Lanes 1-6, after induction with L-arabinose and incubation in SOC for 0, 0.5, 1.0, 1.5, 2.0 and 3.0 hours respectively. The band corresponding to the expected position of the recE and recT proteins is indicated by an arrow.

FIG. 5 is an image of the analysis of false positive recombinant BAC clones. M, Molecular weight marker (MidRange I, New England Biolabs); C, Unmodified pEBAC/148β clone; Lanes 1-5, clones 1-5 (A) Analysis by pulsed field gel electrophoresis after digestion with XhoI; (B) High resolution agarose gel electrophoresis after digestion with EcoRI; (C) Southern blot analysis of (B) using ³²P labelled LUG probe.

FIG. 6 is an image of the analysis of a pEBAC/148β/110 clone demonstrating additionally a deletion of the ^{A}γ-globin gene through intramolecular recombination between the two γ-globin genes. Lane 1, recombinant pEBAC/148β/110 clone; Lane 2, unmodified pEBAC/148β clone. (A) Analysis by pulsed field gel electrophoresis after digestion with XhoI. The arrow points to the 4936 bp γ-globin gene fragment that is deleted in lane 1; (B) High resolution agarose gel electrophoresis after digestion with EcoRI. The arrows point to the 2633 bp and 1585 bp fragments from the γ-globin gene region that are deleted in lane 1; (C) Southern blot analysis of (B) using ³²P labelled LUG probe, demonstrating deletion of the ^{A}γ-globin gene.

FIG. 7 Map of the pGETrec3 plasmid, showing its main features. The tetracycline repressor gene, the I-SceI gene and its recognition site, were inserted as a single fragment at the unique SgrAI site of the pGETrec plasmid. The pGETrec3.1 plasmid was derived from the pGETrec plasmid by removal of the unique I-SceI site.

FIG. 8 General scheme of the two-step GET Recombination system for the insertion of the IVS I-5 and IVS II-654 splicing mutations into the β-globin gene. In the first stage, the 50 base pair regions flanking the I-SceI/Kan^{R} cassette are homologous to the sequences close to the end of IVS I, with only 9 bp gap between the two homology arms. The same first stage construct was used in the second stage, for the insertion of the two different mutations. A 732 bp PCR product from patient DNA was used for the IVS I-5 mutation, while a 1708 bp PCR product encompassing the whole gene was used for the IVS II-654 mutation.

FIG. 9 High resolution fingerprinting of five independent TVS I-5 recombinant clones by EcoRI digestion. A. Lane M: Molecular weight marker; Lane C: pEBAC/148β clone; Lanes 1-5:five individual recombinant clones. The arrow indicates the 5.5 kb fragment on which the IVS I-5 mutation has been inserted. B: Southern blot analysis of the gel depicted in panel A after hybridisation with a ³²P-labebelled LUG probe under conditions of low stringency. The fragments corresponding the each one of the β-globin-like genesare indicated.

FIG. 10 Sequencing chromatograms of recombinant clones carrying the IVS I-5 (A) and IVS II-654 (B) mutations in the β-globin gene.

FIG. 11 Pulse field gel electrophoresis analysis of fault positive recombinant BAC clones generated by deletions of the counterselection cassette and flanking sequences from the DH10B (pEBAC/148β::I-SceI/Kan^{R}, pGETrec3.1) cells. The GET Recombination system was induced for 40 minutes prior to harvesting. Cells were induced by cTc immediately after electroporation with buffer (no DNA) and incubated in SOC for 15, 30, 45 and 60 minutes. At the end of each time point, cells were harvested, BAC DNA was extracted and re-electroporated into normal DM10B cells. DNA was isolated from independent clones, digested with NotI and analysed. M, Low range PFGE molecular weight marker; C, Control pEBAC/148β clone, showing the 185 kb genomic insert and the 16 kb vector; Lanes 1-14, Clones isolated after induction with cTc for 15 minutes (lanes 1-3), 30 minutes (lanes 4-7), 45 minutes (lanes 8-11) and 60 minutes (lanes 12-14).

### DETAILED DESCRIPTION OF THE PREFERRED EMBODIMENTS

The present invention is predicated, in part, on the modification of the GET Recombination system wherein an EcoRI/Kan^{R} cassette is used to facilitate the selection of BAC clones containing human genomic DNA into which have been introduced specific disease causing mutations. These initial findings have now facilitated the development of a counterselection system for selecting nucleic acid molecules which have been modified, in either a non-targeted or a targeted manner, based on use of the induction of double-stranded nucleic acid breaks as the counterselection means such as via the use of restriction endonucleases. The development of the present invention now facilitates, inter alia, a means for accurately and rapidly selecting nucleic acid molecules of interest, in particular BACs or PACs, which have undergone some form of modification, such as a targeted mutation. Accordingly, there is now provided a means of, inter alia, conducting comparative functional analysis of key regulatory elements and developing accurate animal and in vitro cellular models for human genetic disorders.

Accordingly, one aspect of the present invention is directed to a method for selecting a modified nucleic acid molecule or derivative or analogue thereof, said method comprising the steps of facilitating the interaction, in a host cell, of a counterselection marker with an unmodified nucleic acid molecule at a target modification region, which counterselection marker can facilitate the inducible degradation of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the degradation activity of said counterselection marker.

Reference to a “modified” nucleic acid molecule should be understood as a reference to a nucleic acid molecule which has undergone a molecular alteration. By “molecular alteration” is meant a change to the nucleic acid molecule at the level of its component nucleotides. Examples of molecular alterations includes but are not limited to:

(i) Mutating individual nucleotides (eg. substituting an existing nucleotide with an alternative nucleotide or nucleotide analogue).

(ii) Deleting a nucleotide or sequence of nucleotides. The subject deletion should be understood to encompass both deleting one/a few nucleotides or deleting large stretches of nucleic acid sequence.

(iii) Inserting a nucleotide or sequence of nucleotides. This should be understood to include inserting sequences exhibiting functions, for example, as selection marker, counterselection marker, reporter, specific protein folding domain, random mutagenesis cassettes or specific binding sites for regulatory genes.

(iv) Incorporating or deleting isotopically or radioactively labelled nucleic acid sequences.

In accordance with this definition, an “unmodified” nucleic acid molecule is a reference to the form of nucleic acid molecule prior to undergoing one or more of the molecular alterations detailed above. It should be understood that the “unmodified” nucleic acid which is the subject of the method of the present invention is one which was previously modified (eg. to introduce a mutation) but which is to become the subject of further modifications and selection in accordance with the methods defined herein. Accordingly, in this regard, the form of a nucleic acid molecule (whether it be naturally or non-naturally occurring) prior to modification and selection in accordance with the method of the present invention should be understood as corresponding to the “unmodified” form.

The present invention is predicated on the use of a counterselection marker to select for nucleic acid molecules which have undergone a modification of interest. Without limiting the present invention to any one theory or mode of action, the present invention is predicated on the use of a counterselection marker which, upon inducing its degradation activity, facilitates degradation of the nucleic acid molecule with which it is associated. The method developed by the inventors is based on selecting modified nucleic acid molecules by associating a counterselection marker with an unmodified nucleic acid molecule at the site which is targeted for modification. The modification step is then designed such that in introducing the modification of interest the counterselection marker is necessarily deleted. Since the counterselection marker is one which can facilitate degradation of a nucleic acid molecule with which it is associated, inducing the degradation activity of the marker subsequently to modification of a population of nucleic acid molecules will lead to degradation of those nucleic acid molecules which remain associated with the counterselection marker, that is those nucleic acid molecules which have not correctly and/or not completely undergone the desired modification and therefore have not been disassociated with the counterselection marker. Those nucleic acid molecules which have correctly undergone modification, and therefore have seen deletion of the counterselection marker, will largely not be degraded following induction of the counterselection marker's degradation activity.

Accordingly, “counterselection marker” is a reference to a means of selecting for modified nucleic acid molecules. The counterselection marker which is utilised in accordance with the method of the present invention is one which can facilitate the degradation of a nucleic acid molecule with which it is associated.

By “degradation of a nucleic acid molecule” is meant that the subject nucleic acid molecule undergoes the cleavage of one or more of the interactive bonding mechanisms which exist to maintain the structure and/or function of the nucleic acid molecule. Without limiting the present invention to any one theory or mode of action, DNA and RNA are composed of linked nucleotide subunits, which nucleotides comprise a phosphate group linked to a 5 carbon atom sugar which, in turn, is linked to a flat aromatic molecule. A variety of bonding mechanisms are utilised to maintain the structure of a DNA or RNA molecule including, but not limited to, covalent bonds linking the components of each nucleotide subunit, phospodiester covalent bonds between the phosphate group of one nucleotide and the hydroxyl group on the sugar of an adjacent nucleotide and hydrogen bonds between the complementary flat aromatic molecules of two nucleotides (ie., adenine-thymine and guanine-cytosine, for example) in order to maintain the double helix structure of DNA. It should be understood that a variety of other interactive bonding mechanisms may also function to maintain the structure and integrity of a nucleic acid molecule including, but not limited to, van der Waals forces. As detailed hereinbefore, “degradation” of a nucleic acid molecule should be understood as a reference to the cleavage of at least one interactive bond which exists in the nucleic acid molecule.

Still without limiting the present invention in any way, the consequences of cleaving an interactive bond will depend largely on the nature of the bond which is the subject of cleavage and the number of bonds which have been cleaved. Cleavage of non-covalent bonds or of bonds which exist within a nucleotide subunit, rather than those which maintain linkage between nucleotide subunits, may result in destabilisation of a nucleic acid molecule and/or a certain degree of conformational change. Cleavage of a phosphodiester covalent bond linking two nucleotide subunits will result in the breaking of the nucleic acid molecule into two parts. Where the nucleic acid molecule exists as a double helix, such as a BAC, cleavage of a phosphodiester bond in one DNA strand only will lead to a single-stranded break while a cleavage event in each strand will lead to a double-stranded break. Increasing the number of phosphodiester bonds which are cleaved will necessarily increase the number of nucleic acid molecule fragments which are generated. A cleavage event which leads to the fragmentation of a nucleic acid molecule is referred to herein as “cleavage” of the nucleic acid molecule. Preferably, the subject degradation is cleavage.

Accordingly, the present invention more particularly provides a method for selecting a modified nucleic acid molecule or derivative or analogue thereof said method comprising the steps of facilitating the interaction in a host cell, of a counterselection marker with an unmodified nucleic acid molecule at a target modification region, which counterselection marker can facilitate the inducible cleavage of said nucleic acid molecule, facilitating the modification of said nucleic acid molecule at said target modification region, which modification comprises the functional deletion of said counterselection marker from said nucleic acid molecule and selecting the modified nucleic acid molecule wherein said selection step comprises inducing the cleavage activity of said counterselection marker.

Degradation, and in particular cleavage, of a nucleic acid molecule can be achieved by any suitable means. In a preferred embodiment, said cleavage is achieved utilising a nuclease or derivative, homologue, analogue, equivalent or mimetic thereof. Reference to “nuclease” should be understood as a reference to an enzyme which cleaves nucleic acid phosphodiester bonds. The subject nuclease may be any type of nuclease including, but not limited to, a non-specific endonuclease (one which cleaves internal phosphodiester bonds irrespective of the nucleotide sequence at the region of cleavage) or a restriction endonuclease (one which cleaves internal phosphodiester bonds only where a specific nucleotide sequence occurs). Restriction endonucleases are also known as “restriction enzymes”. In a preferred embodiment, the subject cleavage is induced by a restriction enzyme or a derivative, homologue, analogue, equivalent or mimetic thereof. To the extent that it is not specified, reference to a nuclease herein, and in particular an endonuclease or restriction endonuclease, should be understood as a reference to a derivative, homologue, analogue, equivalent or mimetic of said nuclease.

The present invention therefore preferably provides a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said endonuclease.

More preferably, there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible restriction endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said restriction endonuclease.

As detailed hereinbefore, the subject counterselection marker is defined as a means of facilitating the degradation of a nucleic acid molecule with which it is associated, thereby providing a means for selecting a nucleic acid molecule which has been correctly modified, said correct modification being characterised by functional deletion of the counterselection marker with which it was initially associated. By “facilitated” is meant that the subject counterselection marker either directly or indirectly induces, enhances or otherwise contributes to the subject degradation. Without limiting the present invention in any way, reference to a “counterselection marker” is a reference to any means which facilitates the degradation, in particular the cleavage, of a nucleic acid molecule with which it is associated. Counterselection markers suitable for use in the present invention include, but are not limited to:

(i) A nucleic acid molecule encoding an endonuclease, in particular a restriction endonuclease.

(ii) A nucleic acid molecule comprising an endonuclease cleavage site, such as a restriction endonuclease cleavage site.

(iii) A nucleic acid molecule encoding a protein that regulates the activity of an endonuclease.

The counterselection marker of the present invention preferably comprises a nucleic acid sequence component since this may facilitate establishment of a stable interaction of the counterselection marker with an unmodified nucleic acid sequence at a target modification region. It should be understood, however, that the counterselection marker may nevertheless comprise non-nucleic acid components, as required for example, radioactive or isotopic labels. Preferably, the counterselection marker is a nucleic acid molecule encoding an endonuclease, more preferably a restriction endonuclease, or derivative, homologue, equivalent or mimetic thereof or a nucleic acid molecule comprising an endonuclease cleavage site.

Accordingly, in one preferred embodiment, there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence encoding a restriction endonuclease or derivative, homologue, equivalent or mimetic of said restriction endonuclease and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing expression of said counterselection marker.

In another preferred embodiment there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence incorporating a restriction endonuclease cleavage site and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing cleavage at said restriction endonuclease cleavage site.

According to the method of the present invention, the subject counterselection marker facilitates the “inducible” degradation of the nucleic acid molecule with which it is associated. By “facilitates the inducible degradation” is meant that the counterselection marker either directly or indirectly contributes to the subject degradation in an inducible manner. For example, an EcoRI encoding counterselection marker will induce cleavage of nucleic acid molecules at EcoRI cleavage sites upon its expression. In another example, an I-SceI cleavage site counterselection marker provides an I-SceI cleavage site target. However, by “inducible” is meant that the degradation activity of the counterselection marker can be up-regulated or down-regulated. To the extent that the counterselection marker is an endonuclease gene, for example, expression of the gene can be modulated at the transcriptional or translational level. This modulation can be achieved by any one of a number of techniques, including, but not limited to, regulating promoter expression or regulating methylation. For example, the EcoRI encoding counterselection marker exemplified is controlled by the lac promoter. Accordingly, the constitutive expression, in a host cell, of lacl^{q} by the pGETrec2 plasmid suppresses expression of EcoRI. However, in the presence of IPTG, which molecule binds the lacl^{q} expression product and thereby prevents its suppression of the lac promoter, results in up-regulation of EcoRI expression. In another example, to the extent that the counterselection marker is a cleavage site, such as I-SceI, regulation of the cleavage of this region is achieved by modulating intracellular concentrations of the relevant restriction endonuclease, such as I-SceI. Regulating levels of I-SceI, or other relevant restriction endonuclease, may be achieved utilising methods analogous to those described above in relation to modulation of the EcoRI encoding counterselection marker. In the method exemplified herein, the I-SceI gene is incorporated into the pGETrec plasmid, which plasmid is required to facilitate GET Recombination. The plasmid exemplified herein is termed pGETrec3.1 and includes an I-SceI gene which is under tight regulation of the tetracycline repressor. This repressor is inducible by chlorotetracycline. Accordingly, modulation of intracellular levels of chlorotetracycline to regulate I-SceI gene expression which, in turn, regulates cleavage of the I-SceI cleavage site counterselection marker.

Reference to “inducing the degradation activity” of a counterselection marker should therefore be understood as a reference to inducing the marker to contribute, either directly or indirectly, to degradation of the nucleic acid molecule with which it is associated. An example of a direct contribution is one where the counterselection marker expression product interacts directly with the nucleic acid molecule which is the subject of cleavage. An example of an indirect contribution is one where the counterselection marker expression product acts on a molecule other than the nucleic acid molecule which is the degradation target, which molecule itself either directly or indirectly effects degradation of the target nucleic acid molecule. The contribution of the counterselection marker may be active or passive. An active contribution is one where the counterselection marker itself is induced to produce a molecule or signal which directly or indirectly degrades the nucleic acid molecule. An example of an active contribution is one where the subject marker encodes a restriction endonuclease, wherein up-regulation of expression of the marker leads to production of an endonuclease which cleaves the nucleic acid molecule with which the marker is associated. A passive contribution is one where the marker functions as a degradation target. For example, a passive counterselection marker contribution is one where the subject marker comprises a unique restriction endonuclease cleavage site. Although the counterselection marker itself does not produce the degradation signal, it nevertheless provides the target for an appropriate degradation signal. Accordingly, it should be understood that both inducing a counterselection marker to produce a degradation signal, such as the expression of a restriction endonuclease, and the induction of degradation at a counterselection marker cleavage site, are examples of the induction of degradation activity of a counterselection marker within the scope of the present invention.

The present invention is exemplified herein with respect to the use of an EcoRI encoding nucleic acid molecule counterselection marker and an I-SceI cleavage site counterselection marker. In particular, these counterselection markers are exemplified within the context of identifying BACs which have undergone a targeted modification in the DH10B host cell, which modification is effected utilising the GET Recombination system. It should be understood, however, that the counterselection cassettes exemplified herein, and the method defined here in general, is not limited to use with the GET Recombination system. Rather, this method can be applied to any system of achieving homologous recombination including, but not limited to, the GET Recombination system, the recE/recT homologous recombination system or the bacteriophage lambda system.

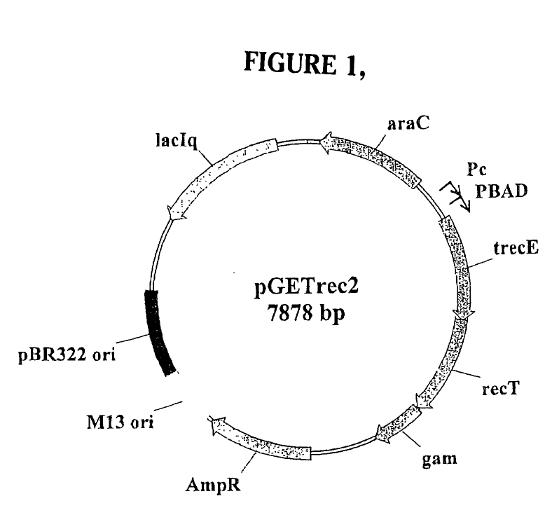

Without limiting the present invention in any way, the large number of EcoRI cleavage sites on the bacterial chromosome and on PAC/BAC clones will result in efficient killing of any cells in which the EcoRI gene is expressed in the absence of the corresponding methylase. As exemplified herein, a modified EcoRI endonuclease gene was placed under the control of the lac promoter. This kills cells expressing lacl^{q} only after induction with IPTG. Since DH10B cells, the host strain for most PAC and BAC libraries, do not normally express lacl^{q}, the lack gene was cloned into the pGETrec plasmid (which plasmid is required to be expressed in the host cell in order to achieve homologous recombination utilising the GET Recombination system) to create pGETrec2 (FIG. 1), a high copy number plasmid, to express lacl^{q} constitutively at high levels. This facilitated the regulated induction of the EcoRI gene by IPTG after GET Recombination, thus enabling the use of the EcoRI gene as a counterselection marker for the modification of PACs and BACs. In the first stage of GET Recombination there was introduced a single copy of the EcoRI/Kan^{R} counterselection cassette into the β-globin gene in a 200 kb β-globin BAC clone. Selection for kanamycin resulted in stable recombinant clones most of which were highly sensitive to IPTG induction. The effective repression of the EcoRI gene by the constitutive expression of the lacl^{q} gene from the pGETrec2 plasmid in DH10B (pEBAC/148β::EcoRI/Kan^{R}, pGETrec2) cells was required for the stable maintenance of BAC clones carrying the EcoRI/Kan^{R} counterselection cassette, since no stable BAC clones could be obtained in the absence of the pGETrec2 plasmid.

In the second stage of GET Recombination, the EcoRI/Kan^{R} cassette was replaced with PCR products carrying only the desired mutations. Introduction of IPTG into the system led to binding of IPTG to lacl^{q} thereby removing the lacl^{q} from its normal binding site on the lac promoter and leading to up-regulation of EcoRI expression due to the loss of lac promoter suppression. Accordingly, BACs which still comprised the EcoRI counterselection marker, indicating that the desired mutation had not been effected, commenced expression of the EcoRI encoding nucleic acid molecule which, in turn, lead to cleavage of the subject BAC at its various EcoRI cleavage sites. Correctly mutated BACs lost the EcoRI counterselection marker and were therefore unaffected by the introduction of IPTG into the system.

Still without limiting the present invention in any way, in another example, an I-SceI endonuclease counterselection system was utilised. Specifically, the I-SceI gene was transferred to the GET Recombination plasmid, pGETrec, to create pGETrec3.1. The counterselection marker comprised the cutting site of I-SceI and, accordingly, a counterselection cassette comprising the I-SceI cutting site and Kan^{R} was inserted, via GET Recombination, into the target modification site of a BAC. The BAC mutation was thereafter inserted at the target modification site utilising the GET Recombination system which functioned via expression of the pGETrec3.1 plasmid. Induction of I-SceI gene expression counterselected BACs which had undergone the desired modification and thereby lost the I-SceI cutting site counterselection marker.

It should be understood that the gene expression of the present invention may take any suitable form such as, but not limited to, episomal or chromosomal expression. Without limiting the invention in any way, the pGETrec2 and pGETrec3.1-based recombination systems function via expression of the regulatory genes in episomal form. However, this system could also function in a highly efficient manner if the relevant genes were expressed after integration on the bacterial chromosome. Such integration could ensure that all cells carry the required genes without the need for antibiotic selection, thereby leading to the production of electrocompetent cells of higher efficiency.

Accordingly, the present invention most preferably provides a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence encoding EcoRI or derivative, homologue, equivalent or mimetic thereof and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing expression of said EcoRI.

In another preferred embodiment there is provided a method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence incorporating an I-SceI cleavage site and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing cleavage at said I-SceI cleavage site.

It should be understood that the counterselection marker of the present invention may be linked, bound or otherwise associated with any other nucleic acid or non-nucleic acid component. For example, to the extent that the counterselection marker is a nucleic acid molecule, it may form part of a nucleic acid counterselection cassette comprising nucleic acid components which provide additional structural or functional attributes. For example, a counterselection marker which is required to be inserted at a target modification region of a nucleic acid molecule may necessarily require the addition of specific flanking sequences in order to facilitate recombination of the counterselection marker at the desired region. Further, it may be desirable to introduce functional features which facilitate the inducible regulation of the degradation activity of the counterselection marker. For example, the EcoRI counterselection marker which is exemplified herein is associated with the lac promoter in order to facilitate the inducibility of its expression via the use of lacl^{q} and IPTG. In another example, both the EcoRI and the I-SceI cleavage site counterselection markers are associated with an antibiotic resistance gene in order to provide a means of quickly and routinely selecting only those nucleic acid molecules which have correctly and stably integrated the counterselection marker, prior to effecting the desired modifications.

As detailed hereinbefore, the counterselection marker functions to facilitate degradation, and in particular cleavage, of the nucleic acid molecule with which it is associated. In this regard, reference to the “interaction” of a counterselection marker with a nucleic acid molecule at a target modification region should be understood as a reference to any form of interaction which associates the marker with the unmodified nucleic acid molecule such that upon inducement of the degradation activity of said marker, degradation of the nucleic acid molecule with which it is associated would be achieved. Means of appropriately associating a particular counterselection marker with a nucleic acid molecule would be well known to those of skill in the art and include, for example, hybridisation between complementary nucleotide base pairs or the formation of bonds between any nucleic acid or non-nucleic acid components of the counterselection marker or the unmodified nucleic acid molecule. Although the preferred method is to cleave the nucleic acid molecule at or around the target modification site and, to the extent that the counterselection marker is a nucleic acid molecule, insert the marker into the cleaved region and reaneal the cleaved ends of the nucleic acid molecule with the ends of the counterselection marker, it may nevertheless be feasible to otherwise associate the counterselection marker with the nucleic acid molecule which is the subject of modification. For example:

(i) For endonucleases that function as complexes of two or more protein subunits or domains, one subunit may be linked to a protein domain specifying a particular DNA sequence recognition specificity while the other domain may be linked to an endonuclease domain. The interaction of the two endonuclease subunits would then be possible only at the DNA site recognised by the first subunit. Such an approach could widen the subject target sites to any sequence which can be recognised by a specific protein domain. Thus combination of this approach with the design of specific Zn finger domains of different sequence specificity is feasible.

(ii) The counterselection marker may comprise a molecule which introduces a cross-link between two DNA strands. For example, the counterselection marker may comprise a peptide nucleic acid molecule which is designed to bind homologously at a region of interest, which peptide nucleic acid molecule is associated with a cross-linking agent which is photoactivated to create a double stranded cross-link. In such a system, the only means by which cross-linked DNA can replicate, and thereby survive, is via excision of the cross-linked region. Where the cross-linked region is not excised, attempts to replicate the linked DNA will ultimately lead to its degradation.

(iii) The counterselection marker may be comprised of specific “padlock” sequences (eg peptide nucleic acids) covalently linked to an endonuclease, this guiding the endonuclease to induce targeted double strand breaks in the region of interest.

(iv) As hereinbefore detailed any method which can be used to introduce double strand breaks in a specific sequence and which is abolished through GET Recombination, for example, can be used as a counterselection marker for the introduction of targeted modifications.

In a particularly preferred embodiment, the counterselection marker is a nucleic acid molecule which is inserted into a target nucleic acid molecule utilising the technique of homologous recombination. Even more preferably, said homologous recombination is the GET Recombination system.

Reference to a “nucleic acid molecule” should be understood as a reference to both deoxyribonucleic acid molecules and ribonucleic acid molecules or derivatives or analogues thereof. The nucleic acid molecules which are utilised in the method of the present invention may be of any origin including naturally occurring (for example, isolated from a biological sample), recombinantly produced or synthetically produced. The subject nucleic acid molecules (ie., the counterselection marker and the nucleic acid molecule which is the target of modification) may be of any form including circular or linear. In this context, a “circular” nucleotide sequence should be understood as a reference to the circular nucleotide sequence portion of any nucleotide molecule. For example, the nucleotide sequence may be completely circular, such as a plasmid, or it may be partly circular, such as the circular portion of the nucleotide molecule generated during rolling circle replication. In this context, the “circular” nucleotide sequence corresponds to the circular portion of this molecule. A “linear” nucleotide sequence should be understood as a reference to any nucleotide sequence which is in essentially linear form. The linear sequence may be a linear nucleotide molecule or it may be a linear portion of a nucleotide molecule which also comprises a non-linear portion such as a circular portion. Examples of linear nucleotide sequences include, but are not limited to, PCR products, excision products, synthesized DNA or the linear portion of a nucleotide molecule generated during rolling circle replication. Preferably, the subject counterselection marker is a linear molecule and the nucleic acid molecule which is the target of modification is a circular nucleic acid molecule. Even more preferably, said circular nucleic acid molecule is an artificial chromosome of the type BAC or PAC. It should be understood that the recombination events which occur in the method of the present invention may occur between nucleotide sequences which are introduced into a cell or they may occur between nucleotide sequences which are naturally found in a cell and one or more introduced nucleotide sequences.

The nucleic acid molecules which are utilised in the method of the present invention are derivable from any human or non-human source. Non-human sources contemplated by the present invention include primates, livestock animals (eg. sheep, pigs, cows, goats, horses, donkeys), laboratory test animal (eg. mice, hamsters, rabbits, rats, guinea pigs), domestic companion animal (eg. dogs, cats), birds (eg. chicken, geese, ducks and other poultry birds, game birds, emus, ostriches) captive wild or tamed animals (eg. foxes, kangaroos, dingoes). reptiles, fish or prokaryotic organisms. Non-human sources also include plant sources such as rice, wheat, maize, barley or canole.

The BAC/PAC cloning system has facilitated the study of the mammalian genome via the gene transfer of large genomic fragments which show correct temporal and tissue-specific gene expression. Large-insert BAC/PAC cloning systems have therefore been used extensively for long-range physical mapping, positional cloning of disease genes, whole genome sequencing projects and functional studies. Accordingly, BAC/PAC E. coli libraries have been created for human genomic DNA as well as for the genomic DNA of other animal and plant species including baboon, canine, bovine, ovine, goat and rice. BACs/PACs are generally maintained at 1-2 copies per cell in the well defined recombination-deficient E. coli strain DH10B. Due to the interest in modifying BACs and PACs, by introducing linear DNA segments, a particularly preferred embodiment of the present invention is directed to the modification of a BAC or PAC via the homologous recombination of a linear DNA segment into the BAC or PAC.

Reference herein to “host cell” should be understood as a reference to any prokaryotic or eukaryotic cell which can be transformed or transfected with a nucleotide sequence. For example, contemplated herein are host cells suitable for cloning and/or expression of nucleotide sequences such as host cells which are used to create gDNA or cDNA libraries or those which are used for cloning a vector which comprises a DNA sequence insert of interest. In accordance with the present invention, which is preferably directed to the targeted modification of BACs and PACs, the host cell is preferably the E. coli strain DH10B.

Accordingly, in one preferred embodiment the present invention provides a method for selecting a modified BAC or derivative or analogue thereof said method comprising the steps of facilitating the interaction, in a DH10B cell, of a counterselection marker with an unmodified BAC at a target modification region, which counterselection marker is a nucleic acid sequence encoding EcoRI or derivative, homologue, equivalent or mimetic thereof and which can facilitate the inducible cleavage of said BAC, facilitating the modification of said BAC at said target modification region, which modification comprises the functional deletion of said counterselection marker from said BAC and selecting said modified BAC wherein said selection step comprises inducing expression of said EcoRI.

In another preferred embodiment there is provided a method for selecting a modified BAC or derivative or analogue thereof said method comprising the steps of facilitating the interaction, in a DH10B cell, of a counterselection marker with an unmodified BAC at a target modification region, which counterselection marker is a nucleic acid sequence incorporating an I-SceI cleavage site and which can facilitate the inducible cleavage of said BAC, facilitating the modification of said BAC at said target modification region, which modification comprises the functional deletion of said counterselection marker from said BAC and selecting said modified BAC wherein said selection step comprises inducing cleavage at said I-SceI cleavage site.

Accordingly, in one preferred embodiment the present invention provides a method for selecting a modified PAC or derivative or analogue thereof said method comprising the steps of facilitating the interaction, in a DH10B cell, of a counterselection marker with an unmodified PAC at a target modification region, which counterselection marker is a nucleic acid sequence encoding EcoRI or derivative, homologue, equivalent or mimetic thereof and which can facilitate the inducible cleavage of said PAC, facilitating the modification of said PAC at said target modification region, which modification comprises the functional deletion of said counterselection marker from said PAC and selecting said modified PAC wherein said selection step comprises inducing expression of said EcoRI.

In another preferred embodiment there is provided a method for selecting a modified PAC or derivative or analogue thereof said method comprising the steps of facilitating the interaction, in a DH10B cell, of a counterselection marker with an unmodified PAC at a target modification region, which counterselection marker is a nucleic acid sequence incorporating an I-SceI cleavage site and which can facilitate the inducible cleavage of said PAC, facilitating the modification of said PAC at said target modification region, which modification comprises the functional deletion of said counterselection marker from said PAC and selecting said modified PAC wherein said selection step comprises inducing cleavage at said I-SceI cleavage site.

“Derivatives” include fragments, parts, portions, mutants, variants and mimetics from natural, synthetic or recombinant sources including fusion proteins. Parts or fragments include, for example, active regions of an endonuclease. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine, aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to nucleic acid molecules or proteins derived from alternative species.

Equivalents of nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dgtu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucinc | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchcp |
| D-α-methyltryptophan | Dmtrp | N-cyclohexylglycine | Nchcx |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcystcine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methyllcucine | Mleu | L-α-methyllysine | Mlys |
| L-α-methylmethionine | Mrnet | L-α-methylnorleucine | Mnlc |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanin | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

As detailed hereinbefore, the method of the present invention is predicated on the positioning of a counterselection marker at a target nucleic acid modification region, modifying the nucleic acid molecule, inducing the degradation activity of the marker and thereby selecting those nucleic acid molecules from which the counterselection marker has been removed by virtue of the modification of the nucleic acid molecule. In terms of the knocking out of the counterselection marker, the present invention exemplifies the use of double stranded PCR fragments. However, it should be understood that other forms of DNA such as synthetic oligonucleotides or denatured PCR fragments comprising homology regions on each side of the counterselection cassette may also be used.

In accordance with the present invention, the degradation activity of the counterselection marker preferably exhibits a predominantly localised effect ie., it preferably acts on the nucleic acid molecule with which it is associated and not, to any significant extent, on neighbouring nucleic acid molecules. Determining the parameters for inducing a counterselection marker to function in a predominantly localised fashion are well within the skills of a person of skill in the art. In this regard, whereas culture conditions can be refined and adapted in order to facilitate essentially localised counterselection marker activity (ie. where the counterselection marker is a restriction endonuclease, minimising degradation of neighbouring non-target nucleic acid molecules which comprise a cleavage site recognised by the subject endonuclease), it should be noted that the use of a counterselection marker which comprises a unique, introduced restriction endonuclease cleavage site is a particularly effective method of ensuring that only nucleic acid molecules comprising this marker can be degraded by cleavage at this site. It should be understood, however, that although the method of the present invention has been designed such that the degradation activity of a counterselection marker functions in a predominantly localised manner, it should nevertheless be understood that there may occur some minor collateral degradation of non-target nucleic acid molecules. As detailed hereinbefore, the incidence of this occurring can be minimised by the person of skill in the art. Without limiting the present invention in any way, the reduction of collateral damage or enhancing specificity may be achieved utilising methods including, but not limited to:

(i) Enhancing specificity of sequence recognition for the target region, for example by the use of a naturally occurring endonuclease with a high sequence specificity or a variant of it designed through directed evolution to exhibit increased specificity for a particular sequence compared to the naturally occurring form.

(ii) Selection/insertion of high affinity recognition sites in the region of interest that do not occur anywhere else in the genome.

(iii) Modulating the conditions for induction of the counterselection marker as well as its half-like to improve overall efficiency.

(iv) Increasing the number of contiguous or near contiguous restriction endonuclease binding sites in the counterselection cassette. For example, although the use of a single site for I-SceI, is exemplified herein, efficiency may be increased by the use of multiple sites.

(v) More effective induction of the restriction endonuclease through the use of alternative promoter systems;

(vi) Reducing the half-life of recE/recT proteins to ensure faster turnover, so as to limit non-specific recombination events that may occur in the cells long after the degradation of the electroporated DNA.

The method of the present invention is predicated on interacting a counterselection marker with an unmodified nucleic acid molecule at a target modification region. By “target modification region” is meant a region of the unmodified nucleic acid molecule which includes at least part of the nucleic acid sequence which is to be modified. For example, the target modification region may comprise all or some of the nucleotides which are to be mutated or deleted. Alternatively, it may comprise the region into which an additional nucleotide sequence is to be inserted. It should be understood, however, that the “target modification region” may define a region which also includes a portion of the nucleic acid molecule which is not the subject of modification. For example, it may be desirable to incorporate the counterselection marker proximally to the site of actual modification. In this case, the region defined by the point of interaction of the counterselection marker through to the site of modification is defined as the “target modification region”. Accordingly, to the extent that a modification of interest will achieve both the introduction of the requisite mutation to the nucleic acid molecule and functional deletion of the counterselection marker, the region spanning the point of interaction of the counterselection marker to the site of mutation comprises the target modification region.

In this regard it should be understood that the “target modification region” encompasses both a region which is specifically identified for modification or a region which is modified as a result of random recombination events. For example, the present invention extends to the induction of deletions arising from illegitimate recombination as well as intramolecular recE/recT promoted recombination between a broken end (produced by nuclease digestion, for example) and other regions of homology on a nucleic acid molecule, such as a BAC, or through mechanisms of non-homologous recombination. In one embodiment, these deletions can be achieved by the induction of the I-SceI counterselection system in the cells in culture with or without the simultaneous induction of recombination (without the need for electroporating a PCR product into the cells).

The modification step which the unmodified nucleic acid molecules undergo is defined as including functional deletion of the counterselection marker which has been introduced at the target modification region. By “functional deletion” is meant that sufficient of the counterselection marker is deleted such that the degradation activity of that counterselection marker cannot be induced. Accordingly, it is not required that the counterselection marker in its entirety is necessarily deleted. In fact, the counterselection marker itself may comprise a nucleic acid component which is intended to remain in the modified nucleic acid molecule as part of the desired modification.

Although the present invention is exemplified in terms of the introduction of a modification at a single target modification region of a nucleic acid molecule, it should be understood that the method of the present invention extends to the introduction of modification at multiple sites on a given nucleic acid molecule. In this regard, it may be desired to modify a nucleic acid molecule, such as a BAC or PAC, at more than one site. Accordingly, the method of the present invention should be understood to extend to the introduction of counterselection markers at more than one site on a nucleic acid molecule. Where the nucleic acid molecule has been correctly modified at each target site, all the counterselection markers will have been removed and the modified nucleic acid molecule will not be degraded. However, where only some of the multiple modifications have occurred, any non-deleted counterselection markers will lead to degradation of the incompletely modified nucleic acid molecule.

It should be understood that the process of the present invention may be homologous or heterologous with respect to the species from which the nucleic acid molecules are derived. A “homologous” process is one where all the nucleic acid molecules utilised in the method of the present invention are derived from the same species. A “heterologous” process is one where at least one of the nucleic acid molecules is from a species different to that of other of the nucleic acid molecules. It should also be understood that in many cases, any given nucleic acid molecule (such as the nucleic acid probe) will not have been derived from any species but will have been designed to comprise a sequence of nucleotides which are not naturally occurring.

In terms of performing the method of the present invention, the steps of:

facilitating the interaction of the counterselection marker with an unmodified nucleic acid molecule,

modifying a nucleic acid molecule (including deleting the counter selection marker); and

inducing the degradation activity of the counterselection maker and thereby selecting modified nucleic acid molecules

may be performed by any suitable method, which methods would be well known to those of skill in the art. Further, it should be understood that the steps of the invention as defined herein are not necessarily required to be performed consecutively. They may be performed in any suitable order as determined by the person of skill in the art. For example, in certain circumstances it may be desirable to perform the modification step and induce the degradation activity of a counterselection marker simultaneously. The desirability and/or appropriateness of performing the method of the present invention in this or in any other manner can be determined by the person of skill in the art with the application of routine procedure.

The method of the present invention should be understood to encompass any form of routine modification or tailoring for a given situation. For example, in an alternative approach, where an endonuclease cleavage site counterselection marker is utilised the nucleic acid molecules which have been subjected to modification in a host cell may be isolated from the host cell following completion of the electroporation step and digested with the restriction endonuclease outside the host cell. Any non-modified molecules will be linearised and destroyed, while modified molecules are re-electroporated into normal host cells.

Still another aspect of the present invention contemplates modified nucleic acid molecules selected by the method of the present invention.

Preferably said modified nucleic acid molecules are modified BACs and modified PACs.

The present invention also extends to the use of said modified nucleic acid molecules in the treatment and/or diagnosis of patients. Methods of treatment include gene therapy regimens. The present invention also extends to methods of screening which utilise said modified nucleic acid molecules.

Accordingly, another aspect of the present invention contemplates a pharmaceutical composition comprising modified nucleic acid molecules generated by the method of the present invention together with one or more pharmaceutically acceptable carriers and/or diluents.

Yet another aspect of the present invention is directed to a kit for facilitating selection of a modified nucleic acid molecule said kit comprising compartments adapted to contain any one or more of a counterselection marker, reagents useful for facilitating modification of a nucleic acid molecule and reagents useful for facilitating selection of said modified nucleic acid molecule. Further compartments may also be included, for example, to receive nucleic acid molecules such as any one or more of the nucleotide sequences which are the subject of modification, the host cells or the nucleic acid molecules required to facilitate the recombination such as that induced by the GET Recombination system, the recE/recT system of recombination or the bacteriophage lambda system.

Further features of the present invention are more fully described in the following non limiting Figures and/or Examples.

### EXAMPLE 1

### TARGETED MODIFICATIONS ON BAC CLONES USING THE GET RECOMBINATION AND AN EcoRI ENDONUCLEASE COUNTERSELECTION CASSETTE

### MATERIALS AND METHODS

Media and Plates

LB medium containing 12.5 mg/ml chloramphenicol (Cm), 100 mg/ml ampicillin (Amp) and 35 mg/ml kanamycin (Kan), was used to grow BAC clones. Following electroporation cells were diluted in SOC medium to allow the expression of antibiotic.

Plasmids and DNA Templates

pEBAC/148β, a BAC clone containing the entire β-globin locus located on a 185 kb genomic insert in a second generation BAC/PAC cloning vector and pGETrec, a 6578 bp plasmid containing the E. coli t-recE and recT genes and the bacteriophage λ gam genes in a polycistronic operon, have been previously described (Narayanan et al., 1999, supra). pGETrec2 plasmid (FIG. 1) was derived from the pGETrec plasmid by the insertion of the lacl^{q} gene at the unique SgrAI site. Briefly, the lacl^{q} gene (bases pairs 3130-4410) from plasmid pGEX-1 Lambda T (U13849) was amplified with primers 5′-TAGTCACACCGGTGCGGCCG-3′ (<400>1) and 5′-GTAGCTCACCGGTGACGTC-3′ (<400>2) and cloned into the unique SgrAI site of pGETrec. pKGS positive-selection vector (Kuhn et al., 1986, supra) was kindly provided by Dr Patricia Green. Genomic DNA from two Thai patients homozygous for the Hb E and the 4 bp deletion mutations respectively was provided by Dr Pranee Fucharoen.

PCR Reactions

PCR primers used for the preparation of the EcoRI/Kan^{R} cassette were obtained from GenSet (Singapore) and were additionally subject to RPC cartridge purification. The cassette was amplified from the pKGS vector using primers:

| EcoKanF | |
|---|---|
| 5′-CAGAGAAGACTCTTGGGTTTCTGATAGGCACTGACTCTCTCTGCCTATTacaagataaaaatatatcat-3′ | (<400>3) |
| EcoKanR | |
| 5′-AAAGAACCTCTGGGTCCAAGGGTAGACCACCAGCAGCCTAAGGGTGGGActtgtttgttgcatttctagcca-3′ | (<400>4) |

Upper case characters (49-50 bp) relate to the homology targeting arms corresponding to sequences in IVS I of the β-globin gene, and lower case characters to those used to prime amplification of the EcoRI/Kan^{R} cassette from plasmid KGS. PCR reactions were performed in 25 μl reactions for 30 cycles (94° C., 30s; 50° C., 30s; 72° C., 2 min) with a high fidelity DNA polymerase (Boehringer Mannheim) using the manufacturer's specifications. PCR products were gel purified to remove template plasmid before use for homologous recombination. PCR colony screening across the recombination junctions was performed with primers

LUG1A 5′-ACAAGACAGGTTTAAGGAGACCA-3′ (<400>5) and LUG2A 5′-GTCTGTTTCCCATTCTAAACTGTA-3′ (<400>6) as previously described (Narayanan et al. 1999, supra). These primers yield a PCR product of 447 bp from the normal β-globin gene, while insertion of the EcoRI/Kan^{R} cassette should yield a PCR product of 2.6 kb. The same primers were used for preparation of the LUG probe for Southern blot hybridisations. PCR using ARMS1 5′-TACGGCTGTCATCACTTAGACCTCACCCTG-3′ (<400>7) and LUG2A primers was used to amplify a 732 bp product from genomic DNA of patients carrying the Hb E mutation and a 728 bp product for the 4 bp deletion mutation.

Sequencing Reactions

Direct DNA sequencing of recombinant BAC clones was performed with the Big Dye Terminator kit (Perkin Elmer) using the protocol recommended by the manufacturer.

Homologous Recombination

The first round of GET Recombination was performed as previously described (Narayanan et al. 1999, supra). For the second stage, approximately 0.3 μg of purified PCR product was electroporated (Bio-Rad “Gene-Pulser II”) into 30 μl of electrocompetent E. coli DH10B (pEBAC/248b::EcoRI/Kan^{R}, pGETrec2) cells using 1.8 KV/cm, 200 Ohm, 25 μF. The cells were prepared for electroporation by growing 250 ml cultures, inoculated from an overnight culture at 30° C., in LB containing chloramphenicol, ampicillin and kanamycin until an OD₆₀₀ of 0.4-0.5 was reached. Expression of the recE, recT and the gam genes was then induced by the addition of L-arabinose to a final concentration of 0.2% (w/v) for a further 40 minutes at room temperature. The cells were then harvested and made electrocompetent by standard methodoloy. Induction of the EcoRI endonuclease gene for counterselection was done either on LB plates containing chloramphenicol and 1 mM IPTG, or by adding 1 mM IPTG to SOC after 3 hours of incubation at 37° C. for a further 2 hours, before plating on LB plates containing chloramphenicol and 1 mM IPTG. Single colonies were grown overnight in LB containing chloramphenicol and 1 μl aliquots were used for PCR screening for recombinant clones. To rescue modified BAC clones from the pGETrec2 plasmid, cells were plated after overnight growth in the absence of ampicillin. Colonies that lost the pGETrec2 plasmid were readily identified by replica plating on Cm and Cm/Amp plates.

Analysis of BAC DNA

BAC DNA was purified using a standard alkaline lysis protocol for BACs (Osoegawa, K., de Jong, P. J., Frengen E., Ioannou, P. A. (1999) Construction of Bacterial Atificial Chromosome (BAC/PAC) Libraries, in Current Protocols in Human Genetics, Unit 5.15 Eds Dracopoli, N. C., Haines, J. L., Korf, B. R., Moir, D. T., Morton, C. C., Seidman, C. E., Seidman, J. G., Smith, D. R. John Wiley & Sons, New York) or the CsCl ultracentrifugation method. For gel analysis 0.5 μg of DNA was digested with 1-2 units of EcoRI, NotI, or XhoI. The products of NotI and XhoI digestion were size fractionated using CHEF under the following conditions: 1% gel, in 0.5×TBP buffer, at 14° C., 6 V/cm for 16 hr, with 1-20 sec pulse time at a 120° angle. Analysis of EcoRI digests was on a 1% gel in 0.5×TBE at room temperature, 4 V/cm for 8 hours. The gels were stained with ethidium bromide for visualisation.

Hybridization

After separated by agarose gel electrophoresis DNA was transferred onto nylon membrane (Hybond N+, Amersham) using the Southern blotting alkaline transfer method (Sambrook, J., Fritsch, E. F. and Maniatis, T. (1989) Molecular cloning: A laboratory manual, 2^{nd} ed. Cold Spring Harbor Lab. Press. Cold Spring Harbor). Membranes were placed in 0.25M Na₂HPO₄ buffer (pH 7.2), 7% SDS, 1% BSA and 1 mM EDTA. ³²P-labelled LUG probe was added to the hybridization solution and hybridization was performed at 65° C. overnight in a rotating oven. Washing was carried out with 2×→0.2×SSC, 0.1% SDS at 65° C.

Analysis of in Vivo Stability of the recE and recT Proteins

The RecE and RecT proteins were analysed by discontinuous SDS-PAGE by the method of Laemmli, U. K. (1970) Nature 227:680-685. A whole cell protein extract was prepared from 40 μl of electrocompetent cells, with and without induction with 0.2% w/v L-arabinose. Similarly a protein extract was also made from equal amounts of electrocompetent cells after incubation for various time points in SOC at 37° C. Samples for SDS-PAGE analysis were prepared by boiling appropriate aliquots of cells for 20 minutes in SDS-PAGE sample buffer. After allowing them to cool to room temperature, they were briefly centrifuged to remove particulate matter, and the resulting denatured protein extracts were resolved on 10% polyacrylamide gels using a Protean II minigel apparatus (Bio-Rad). Visualisation of polypeptides was performed by Coomassie Blue staining.

### EXAMPLE 2

### EcoRI ENDONUCLEASE GENE AS A COUNTERSELECTION MARKER

The EcoRI endonuclease gene is highly toxic to E. coli cells in the absence of the corresponding methylase gene (Betlach, M., Hershfield, V., Chow, L., Brown, W., Goodman, H. and Boyer, H. W. (1976) Fed. Proc. 35-2037-2043). However, a mutant EcoRI endonuclease gene under the control of the lac promoter was isolated which killed cells only after induction with IPTG. A positive-selection vector, pKGS, was thereby developed, containing the mutant EcoRI endonuclease gene and the kanamycin resistance (Kan^{R}) gene (Kuhn et al., 1986, supra). This plasmid has been used as the template for the PCR amplification of the EcoRI/Kan^{R} counterselection cassette.

In preliminary experiments the pKGS plasmid was shown to be highly toxic to DH10B cells, the host E. coli strain in which most PAC and BAC libraries are maintained. Since DH10B cells do not normally express the lacl^{q} gene, the constitutive expression of the EcoRI gene from the pKGS plasmid in these cells caused instability of the plasmid and the only clones that could be isolated had rearrangements of the pKGS plasmid, with inactivation of the EcoRI gene. In order to enable regulation of the expression of the EcoRI endonuclease gene in DH10B cells during GET Recombination, the lacl^{q} gene was therefore cloned from plasmid pGEX-1 into the SgrAI site of the pGETrec plasmid (Narayanan et al., 1999, supra), to produce plasmid pGETrec2 (FIG. 1). The pGETrec2 plasmid was then electroporated into DH10B (pEBAC/148β) cells, carrying a 200 kb BAC clone with the intact β-globin locus (Narayanan et al., 1999, supra), and electrocompetent cells were prepared for the first round of GET Recombination. In order to facilitate targeting of the EcoRI/Kan^{R} cassette into the β-globin gene, the EcoRI/Kan^{R} cassette was amplified by PCR from the pKGS plasmid with primers EcoKanF/EcoKanR, to produce a approximately 2.2 kb PCR product. Insertion of the cassette was targeted into intron I of the β-globin gene, with the deletion of 9 bp in between the homology regions. In the first round of GET Recombination DH10B (pEBAC/148β, pGETrec2) electrocompetent cells were transformed with about 0.3 μg of the 2.2 kb PCR product, and recombinants were selected by plating on agar containing kanamycin and chloramphenicol. Twenty colonies were picked and screened by PCR for the insertion of the EcoRI/Kan^{R} cassette using primers LUG1A and LUG2A. These primers are located outside of the region of homologous recombination and normally amplify a 447 bp product, while correct insertion of the EcoRI/Kan^{R} cassette should yield a approximately 2.6 kb PCR product. Twelve of these colonies were positive for the presence of the 2.6 kb product. Sequencing of the 2.6 kb PCR product with the LUG1A/LUG2A primers demonstrated the precise homologous recombination of the EcoRI/Kan^{R} cassette into the β-globin gene target sites (data not shown). The remaining colonies were negative for both the 447 bp and 2.6 kb PCR products. Because of the extensive homologies that exist between the globin genes, it is likely that these clones may have arisen by recombination at other homologous sites (Orford et al., 2000, supra).

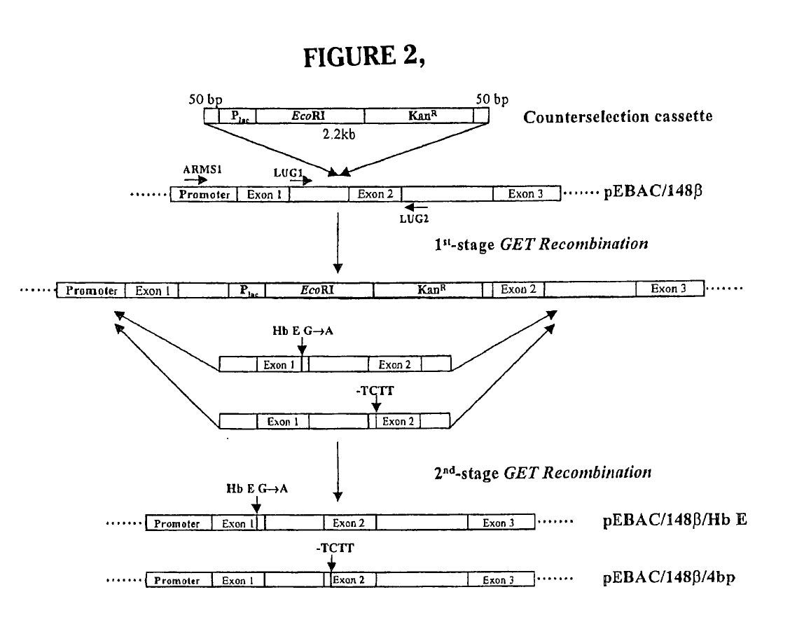

Individual DH10B (pEBAC/148β::EcoRI/Kan^{R}, pGETrec2) recombinant clones were tested for the efficiency of killing by the EcoRI endonuclease gene by plating for 16 hours on agar containing chloramphenicol/kanamycin and 1 mM IPTG. Most of the clones showed a 10⁶-10⁷ fold reduction of the number of colonies on 1 mM IPTG, therefore demonstrating the insertion of a fully functional EcoRI/Kan^{R} cassette into the β-globin locus. The deletion of the counterselection cassette in the second round of GET Recombination may be used to introduce anyone of a number of different modifications in the targeted region and flanking sequences. This was demonstrated in two different experiments where the same pEBAC/148β::EcoRI/Kan^{R} construct was used to introduce two different disease-causing mutations into the β-globin gene (FIG. 2).

### EXAMPLE 3

### INTRODUCTION OF THE HbE MUTATION

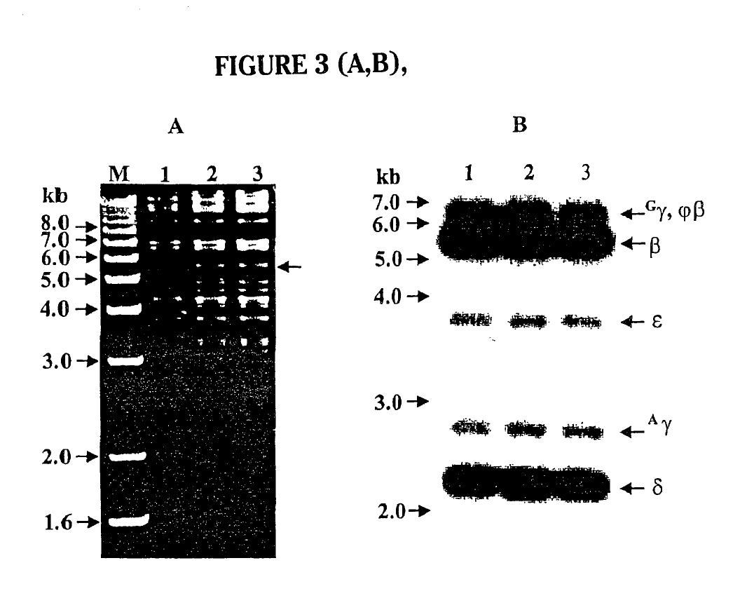

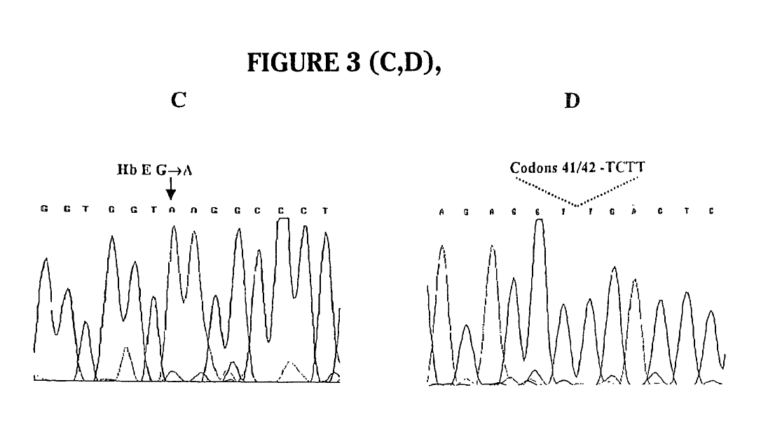

A 732 bp ARMS1-LUG2A PCR product amplified from genomic DNA of a patient homozygous for the HbE (codon 26, GAG→AAG) mutation was first transformed into DH10B (pEBAC/248β::EcoRI/Kan^{R}, pGETrec2) electrocompetent cells using the GET Recombination protocol. After 1 hour of induction in SOC medium at 37° C., recombinant clones were plated on agar containing chloramphenicol and 1 mM IPTG. A large number of clones were obtained under these conditions. Correct excision of the EcoRI/Kan^{R} cassette from the β-globin gene by the genomic PCR fragment should restore the original size of the PCR product with primers LUG1A LUG2A (i.e. 447 bp). Screening of 570 colonies by PCR with primers LUG1A/LUG2A for the deletion of the EcoRI/Kan^{R} cassette yielded only two clones (0.35%) that appeared to have the expected 447 bp PCR product. Restriction analysis of these clones with NotI and XhoI revealed no unwanted changes or rearrangements in any of the restriction fragments. High resolution analysis of an EcoRI digest similarly indicated the absence of any unwanted rearrangements (FIG. 3A, lanes 2-3). Southern blot analysis of the EcoRI digest using the PCR product from primers LUG1A/LUG2A as probe under conditions of low stringency confirmed the absence of any unwanted modifications in any of the globin genes (FIG. 3B). Finally sequencing of the recombinant BAC clones confirmed the presence of the Hb E mutation at the expected position in the β-globin gene, without any other sequence changes in the regions of recombination (FIG. 3C).

Given the high efficiency of killing of DH10B (pEBAC/148β::EcoRI/Kan^{R}, pGETrec2) cells by induction of the EcoRI endonuclease gene the large number of false positive clones was somewhat surprising. It was also further noted that most of the false positive clones failed to grow on kanamycin or give any PCR product with LUG1A/LUG2A primers, indicating that the EcoRI/Kan^{R} counterselection cassette was deleted and that this was associated with deletion of additional sequences at one or both ends of the cassette. This was in contrast to the results obtained previously with the tetracycline counterselection cassette (Nefedov et al., 2000, supra), where most false positive clones appeared to have an intact tetracycline resistance gene. Since the initial testing of the killing efficiency of the EcoRI endonuclease gene on DH10B (pEBAC/148β::EcoRI/Kan^{R}, pGETrec2) cells was carried out without induction of the GET Recombination system with L-arabinose, these results indicated that the GET Recombination system was facilitating the rescue of some of the plasmid molecules that were linearised by EcoRI endonuclease before they were totally degraded.

### EXAMPLE 4

### INTRODUCTION OF THE CODONS 41/42 (-TTCT) DELETION

A modified counterselection approach was therefore used in a further experiment to introduce the codons 41/42 (-TTCT) deletion into the β-globin gene. The PCR product obtained with the ARMS1-LUG2A primers from the genomic DNA of a patient homozygous for this deletion was electroporated into E. coli DH10B (pEBAC/148β::EcoRI/Kan^{R}, pGETrec2) cells and the cells were incubated for 3 hours in SOC at 37° C., before induction of EcoRI by the addition of 1 mM IPTG and a further incubation for 2 hours prior to plating on LB agar plates supplemented with chloramphenicol and 1 mM IPTG. It was anticipated that the prolonged incubation of cells in SOC prior to induction of EcoRI would allow the recE and recT proteins to turn over more effectively, thus reducing the chances of any rescue of plasmid molecules linearised by EcoRI endonuclease. PCR screening of 90 clones with primers LUG1A/LUG2A identified 4 positive clones (4.44%), indicating a 10-fold enhancement of counterselection efficiency under these conditions. Analysis of these clones by digestion with NotI, XhoI and EcoRI and by Southern blot analysis after EcoRI digestion did not show any unwanted rearrangements, while sequencing confirmed the insertion of the 4 bp deletion into the β-globin gene (FIG. 3D).

### EXAMPLE 5

### ANALYSIS OF FALSE POSITIVE CLONES

In the above experiments the number of clones surviving EcoRI endonuclease counterselection was much greater than anticipated given the efficiency of the GET Recombination system (Narayanan et al., 1999, supra) and the low rate of resistant clones that may arise from spontaneous mutations in the EcoRI endonuclease gene. The results indicated an interaction between the two systems) whereby the ends of fragments produced by EcoRI endonuclease digestion could become substrates for the GET Recombination system. In the simplest situation, a linearised plasmid molecule with a single double strand break could be rescued from total degradation by intramolecular recombination between a repetitive sequence close to one end and other homologous sequences on the plasmid. Alternatively, double strand breaks could also be repaired by a variety of non-homologous recombination mechanisms.

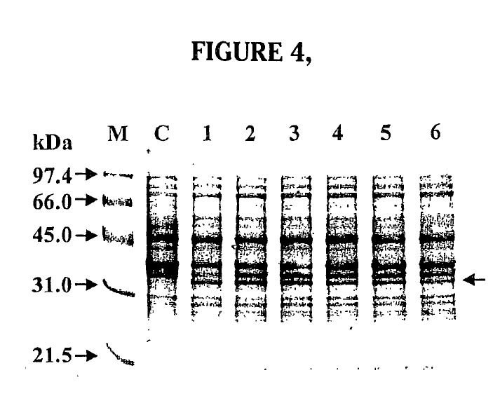

The time course of disappearance of the RecE and RecT proteins during incubation in SOC was therefore examined in whole cell extracts (FIG. 4). The RecE (280 amino acids) and RecT (269 amino acids) proteins are expected to be very similar in size (about 30-31 kDa) and are not resolved on electrophoresis of extracts from electrocompetent cells (FIG. 4). Prolonged incubation of electrocompetent cells in SOC for up to three hours was found to result only in a limited reduction of the intensity of the RecE/RecT band (FIG. 4). It is not clear at this stage if both the RecE and RecT proteins decay at the same rate. The long persistence of one or both of these proteins clearly indicates, however, the possibility of significant RecE/RecT-dependent deletions after induction of EcoRI and could account for the high frequency of deleted clones that were observed in this experiment.

In order to gain an understanding of the relative contribution of the different possible pathways for the generation of false positive clones, analysis of false positive clones was initially carried out by PCR using primers outside the targeting cassette. About 2-5% of IPTG-resistant clones retained resistance to kanamycin and gave a PCR product of 2.6 kb, indicating that the EcoRI/Kan^{R} cassette was basically intact. Such clones may arise from spontaneous mutations in the EcoRI gene and account for only a small fraction of the false positive clones.

In some clones the deletion of the EcoRI cassette was accompanied with the appearance of a larger PCR fragment, indicating some type of insertion. Sequence analysis of one such clone revealed the insertion of the Tn10 transposon gene between exons 1 and 3 of the β-globin gene (data not shown). This presumably was mobilised from the bacterial genome to reseal the ends of a BAC clone resulting from an abortive recombination event in the β-globin gene and points to an important role of transposons as a defence mechanism for the repair of double strand breaks.

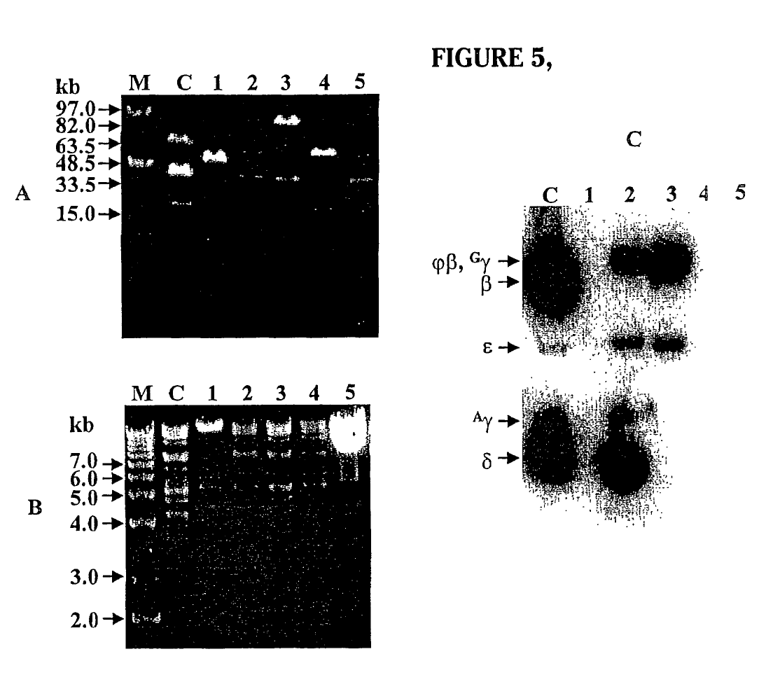

The majority (90-95%) of the false positive clones failed to give any PCR product with flanking primers, indicating that one or more recombination events had taken place that had damaged one or both primers bindings sites, with the simultaneous inactivation or deletion of the EcoRI gene. Analysis of clones by XhoI digestion indicated the presence of a large variety of deletions (FIG. 5A), yet independent clones with apparently the same deletions were also identified in separate experiments. A large deletion of about 150 kb is obvious in clone I after XhoI digestion, (FIG. 5A), while partial or complete deletions of one or more fragments and the appearance of new fusion fragments are also obvious after EcoRI digestion in the other clones (FIG. 5A, clones 2-5). Deletions of various fragments are also obvious by ethidium bromide staining after EcoRI digestion. In some of these clones, the number of EcoRI fragments is dramatically reduced (FIG. 5B, clones 1 and 5). Southern blot analysis of the EcoRI gel with probe LUG1A/LUG2A showed complete deletion of all the globin gene sequences in some clones (FIG. 5C, clones 1, 4 and 5), while other clones showed deletion of the β-globin gene and one or more of the other globin gene sequences (FIG. 5C, clones 2, 3). Most deletions in false positive clones appear to be associated with a single recombination event initiated at the β-globin gene and involving the deletion of variable amount of sequence together with the EcoRI/Kan^{R} cassette.

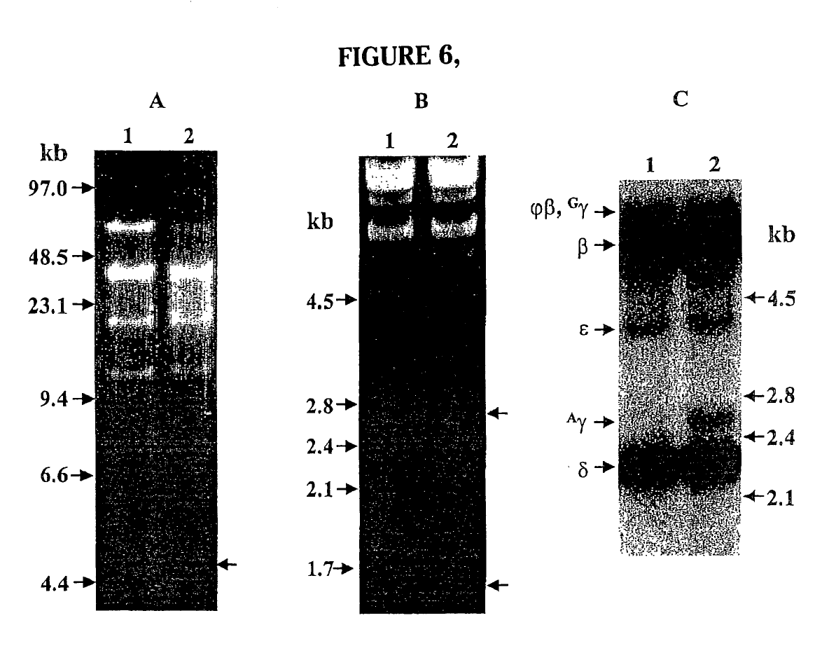

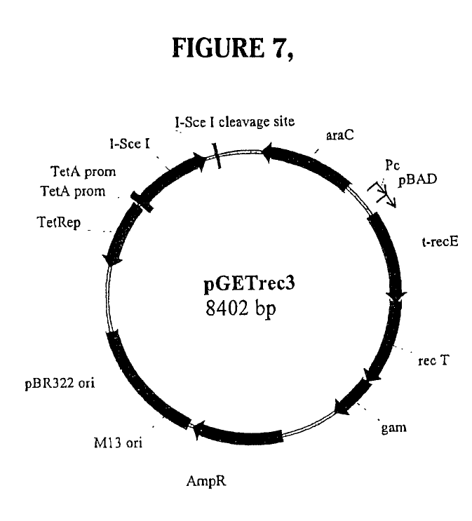

Evidence for the occurrence of two independent recombination events in a single BAC molecule was also obtained in a clone isolated in an experiment involving the introduction of the IVS I-110 G→A splicing mutation into the β-globin gene. Thus while the EcoRI/Kan^{R} cassette appeared to be accurately replaced by a PCR fragment carrying the IVS I-110 G→A splicing mutation, this clone appeared to have an additional deletion of the 4936 bp fragment produced by XhoI digestion between the two γ-globin genes (FIG. 6A), while the other fragments appeared unaffected. Analysis after EcoRI digestion (FIG. 6B) confirmed the deletion of the 2633 bp and 1585 bp fragments from the γ-globin gene region, while Southern blot analysis (FIG. 6C) confirmed the deletion of the 2633 bp ^{A}γ-globin gene fragment, without deletion of the δ-globin gene from the intervening region (FIG. 6). This deletion presumably arose by an intramolecular RecE/RecT dependent recombination event between the two highly homologous γ-globin genes, after the introduction of a double-strand break by EcoRI endonuclease at one of the EcoRI cutting sites that are located between the two genes and is analogous to a naturally occurring form of γ-thalassaemia (Sukumaran,, P. K., Nakatsuji, T. Gardiner, M. B., Reese, A. L., Gilman, J. G. and Huisman, T. H. Nucleic Acids Res. 11:4635-4643).

### EXAMPLE 6

### AN EFFICIENT SYSTEM FOR TARGETED MODIFICATIONS OF BAC CLONES USING GET RECOMBINATION AND AN I-SceI COUNTERSELECTION CASSETTE

### MATERIALS AND METHODS

Media

Standard laboratory media (LB, SOC) and agar plates were used. Antibiotics were used at the following concentrations: Chloramphenicol (Cm) 12.5 μg/ml; Ampicillin (Amp) 100 μg/l; Kanamycin (Kan) 25 μg/ml. Heat-treated chlorotetracycline (cTc) was used to inactivate the tetracycline repressor. The inducer cTc was suspended in LB medium at a concentration of 400 μg/ml and autoclaved for 20 min, then stored in the dark. Induction of the I-SceI gene for counterselection was carried out by adding cTc stock into SOC medium and LB agar plates at final concentration 50 μg/ml.

Plasmids and DNA Templates

pST98AS, an inducible I-SceI-expressing plasmid under the control of the tetracycline promoter and repressor has been previously described (Posfai, G. et al. (1999) Nucleic Acids Res. 27 4409-4415). pST98AS/Kan plasmid was derived from the pST98AS plasmid by the insertion of the kanamycin resistance gene (Kan^{R}) into the NcoI site, downstream of the I-SceI recognition site. The kanamycin resistance gene was amplified from the pZero™-₂ plasmid (Invitrogen) with primers NcoIKanF 5′-CATGCCATGGTCAAGAAATCACAGCCGAA-3′ <400>8 and NcoIKanR 5′-CATGCCATGGCGTGATCTGATCCTTCAAC-3′ <400>9, and cloned into the NcoI site of pST98AS, to produce plasmid pST98AS/Kan. This plasmid was used as the template for amplification of the I-SceI/Kan^{R} cassette.

pGETrec, a 6578 bp plasmid containing the E. coli t-recE and recT genes and the bacteriophage λ gam gene in a polycistronic operon, has been previously described (Narayanan et al, 1999, supra). pGETrec3 was derived from the pGETrec plasmid by the insertion of the I-SceI gene, together with its recognition sequence and the tetracycline repressor gene, at the unique SgrAI site (FIG. 1). The I-SceI gene cassette was amplified from the pST98AS plasmid with primers

| ISccTetF | 5′-TAGTCACACCGGTGGTTAACTCGACATCTTGG-3′ |
|---|---|
| <400>10 and | |
| ISceR | 5′-GTAGCTCACCGGTGCAATGTAACATCAGAGA-3′ |
| <400>11 | |

and cloned into the SgrAI site of the pGETrec plasmid. pGETrec3.1 was derived from pGETrec3 by digestion with I-SceI, polishing of the ends with T4 polymerase and blunt end ligation, so as to destroy the unique I-SceI recognition site.

pEBAC/148β, a second-generation BAC/PAC clone containing the entire β-globin locus located on a 185 kb genomic insert has been previously described (Narayanan et al, 1999, supra). Genomic DNA from two Thai patients homozygous for the IVS 1-5 (G→C) and IVSII-654 (C→T) mutations respectively was provided by Dr. Pranee Fucharoen.

Preparation of Linear DNA Cassettes

Standard PCR conditions were used to amplify linear DNA fragments with the Expand High Fidelity PCR system (Boehringer Mannheim). For the first-stage recombination (FIG. 2), the I-SceI/Kan^{R} cassette, including the tetracycline repressor gene, was amplified from the pST98AS/Kan plasmid with the primers

| I-SccIKanF | 5′-CAGAGAAGACTCTTGGGTTTCTGATAGGCACTGACT |
|---|---|
| | GTCTCTGCCTATTtatcagggttattgtctcatg-3′ |
| | <400>12 and |
| I-SceIKanR | 5′-CAAAGAACCTCTGGGTCCAAGGGTAGACCACCAGCA |
| | GCCTAAGGGTGGGAggcgtgatctgatccttcaac-3′ |
| | <400>13, |

yielding a 3026 bp PCR product. Upper case characters correspond to the homology targeting arms of the globin genomic sequences, while lower case characters correspond to the sequences used to prime amplification of the I-SceI/Kan^{R} cassette. For the second-stage 3 recombination (FIG. 2), the genomic DNA fragment carrying the IVS I-5 (G→C) mutation was amplified from patient DNA with the primers

| ARMS1 | 5′-TACGGCTGTCATCACTTAGACCTCACCCTG-3′ |
|---|---|
| | <400>7 and |
| LUG2A | 5′-GTCTGTTTCCCATTCTAAACTGTA-3′ |
| | <400>6, |

yielding a 732 bp PCR product. Similarly, the DNA fragment carrying the IVS II-654 (C→T) mutation was amplified with primers ARMS1 and HbbRev 5′-GGCAGAATCCAGArGCTCAA-3′ <400>14, yielding a 1708 bp product. PCR products were gel purified using the Qiagen PCR purification kit, concentrated by ethanol precipitation and suspended in 0.5×TE buffer for electroporation.

Preparation of Electrocompetent Cells and Electroporation

Electrocompetent cells were prepared by inoculating 200 μl of an overnight E. coli culture into 200 ml LB medium with an appropriate antibiotic at 30° C. to an OD₆₀₀ of 0.4-0.5. Expression of the recE, recT and gam genes was then induced by the addition of L-arabinose to a final concentration of 0.2% (w/v) for a further 40 minutes at room temperature. Cells were centrifuged at 5000 rpm for 10 min at 4° C., and washed three times with ice-cold 10% glycerol. The final cell pellet was resuspended in 400 μl of ice-old 10% glycerol. Aliquots (30 μl) were frozen in liquid nitrogen and stored at −70° C. For electroporation, cells were thawed on ice and mixed with purified PCR product. Electroporation was performed in a 0.1 cm cuvette with a Bio-Rad Gene Pulser set at 1.8 kV, 25 μF, and with the pulse controller set at 200 ohms. Cells were immediately diluted with 1 ml SOC medium, incubated at 37° C. for 1 hour and plated onto selective medium unless otherwise specified.

PCR Screening of Recombinant Clones

PCR colony screening across the recombination junctions was performed with primers: LUG1A 5′-ACAAGACAGGTTTAAGGAGACCA-3′ <400>5 and LUG2A. These primers yield a PCR product of 447 bp from the normal β-globin gene, while insertion of the I-SceI/Kan^{R} cassette yields a PCR product of 3365 bp. The same primers were used for the preparation of a β-globin LUG probe for Southern blot hybridisations, as well as for the detection of the recombinant clones carrying the IVS I-5 mutation.

### Sequencing Reactions

Direct DNA sequencing of the recombinant BAC clones was performed using Big Dye Terminator kit (PE Applied Biosystems, Foster City, Calif.) using the manufacturer's protocol.

### EXAMPLE 7

### INSERTION OF THE I-SCEI/KAN^{R} CASSETTE INTO THE β-GLOBIN GENE

Most of the common disease-causing mutations in the β-globin gene are located in the 5′ portion of the gene. Thus in order to facilitate the insertion of some of these mutations into the β-globin locus, the I-SceI/Kan^{R} cassette was amplified from the pST98AS/Kan plasmid and inserted into intron 1 of the β-globin gene with the deletion of only 9 bp between the target homology regions. The first-stage GET Recombination reaction was carried out by electroporation of about 1 μg of the purified I-SceI/Kan^{R} PCR product into E. coli DH10B (pEBAC/148β, pGETrec) cells Recombinant clones were selected by plating the cells onto LB agar plates containing chloramphenicol and kanamycin. Thirteen colonies were picked and screened by PCR for the insertion of the I-SceI/Kan^{R} cassette using the primers LUG1A and LUG2A. Correct insertion of the I-SceI/Kan^{R} cassette should yield a 3365 bp product with these primers, in contrast to the 447 bp product obtained from the unmodified gene. Three of these colonies were positive for the 3365 bp product. Restriction analysis of these clones did not show any unwanted rearrangements, while direct sequencing with the LUG1A/LUG2A primers demonstrated the precise homologous recombination of the I-SceI/Kan^{R} cassette into the β-globin gene target site (data not shown).

### EXAMPLE 8

### USE OF THE I-SCEI GENE AND ITS RECOGNITION SITE FOR COUNTERSELECTION

To test the efficiency of killing of non-recombinant clones by the I-SceI/Kan^{R} cassette, DH10B (pEBAC/148β::I-SceI/Kan^{R}, pGETrec) cells were plated onto LB agar plates containing chloramphenicol, ampicillin and cTc. Induction of the I-SceI gene is expected to generate a DSB at the unique I-SceI recognition site, leading to degradation of the BAC DNA and inability of the host cell to grow on chloramphenicol. However, we found that the number of colonies on cTc plates was not significantly different from the number of colonies on plates without cTc, indicating that expression of I-SceI endonuclease from the single copy I-SceI gene was too low to induce DSBs and prevent colony formation, and/or that the DH10B cells can effectively repair double strand breaks in BAC clones. To overcome this problem we attempted to increase the I-SceI gene dosage in the cells by the insertion of the I-SceI gene onto the multicopy pGETrec plasmid, to produce the pGETrec3 plasmid (FIG. 1). The I-SceI recognition sequence was also included in the pGETrec3 plasmid, in order to facilitate loss of the pGETrec3 plasmid from the cells after recombination. However, this modification was still ineffective in the killing of the DH10B (pEBAC/148β::I-SceI/Kan^{R}, pGETrec3) cells. Presumably the presence of the I-SceI recognition site on the pGETrec3 plasmid limited its copy number alter induction of I-SceI by cTc. In an effort to increase further the I-SceI gene dosage, the I-SceI recognition site on pGETrec3 was destroyed, to produce pGETrcc3.1. With this modification, induction of I-SceI gene expression by plating DH10B (pEBAC/148β::I-SceI/Kan^{R}, pGETrec3.1) cells on cTc resulted in approximately 1000-fold reduction in the number of surviving colonies compared to plates without cTc, thus indicating that the counterselection system was working with a relatively low efficiency.

The efficiency of killing of DH10B (pEBAC/148β::I-SceI/Kan^{R}, pGETrec3.1) cells after induction of I-SceI endonuclease by cTc was also evaluated in mixed cultures with DH10B (pEBAC/148β, pGETrec3.1) cells, carrying the unmodified BAC clone. Growing of mixed cultures (1:1) in the presence of cTc resulted in the DH10B (pEBAC/148β, pGETrec3.1) cells comprising more than 95% of all cells after 10 generations, demonstrating a strong selective advantage for the cells without an I-SceI recognition site on the globin BAC (data not shown). Since such cells are essentially identical to cultures of clones in which the I-SceI/Kan^{R} cassette is knocked out, these studies indicated that the efficiency of counterselection could be considerably enhanced by exposure of cells to cTc prior to plating.

### EXAMPLE 9

### INTRODUCTION OF THE IVS I-5 (G→C) MUTATION

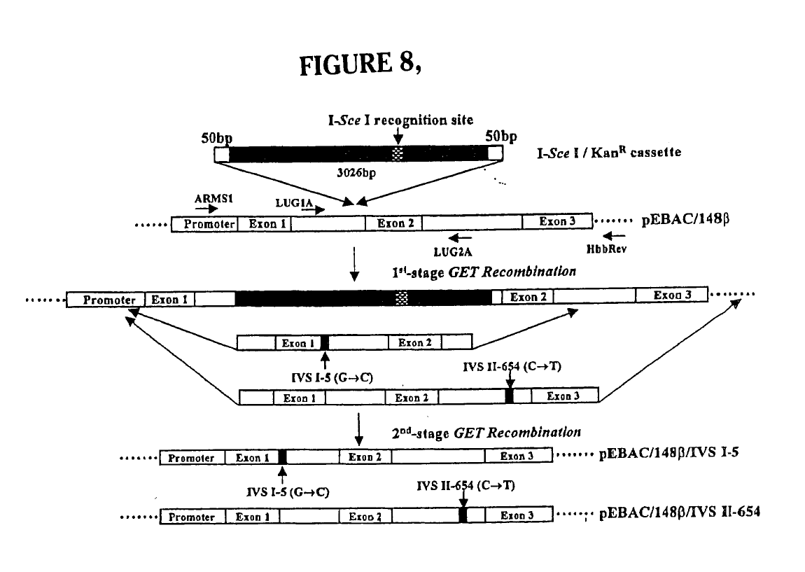

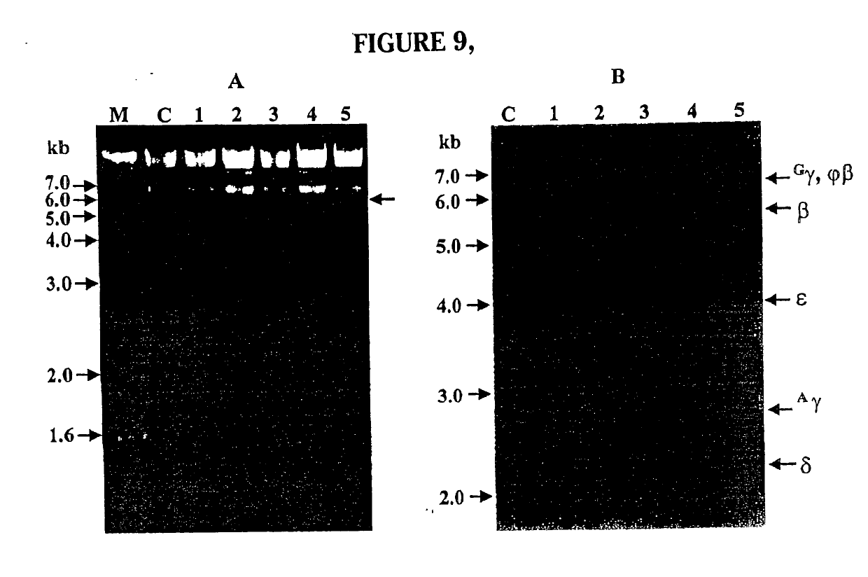

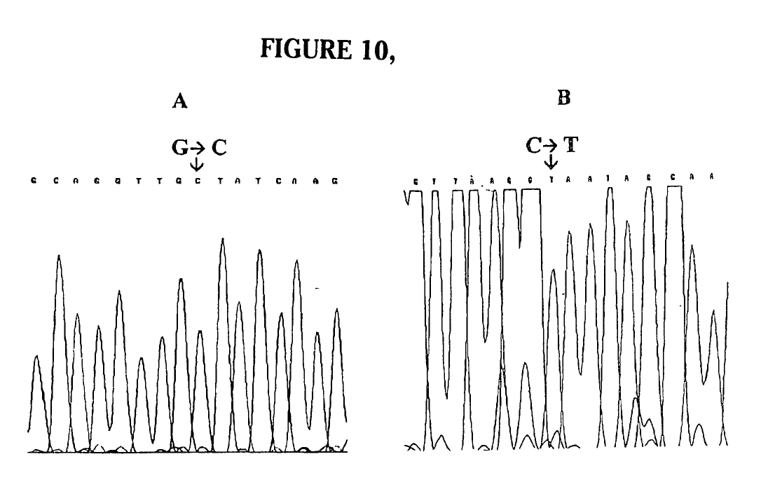

The main steps of the two-stage GET Recombination procedure for the introduction of desired mutations into BAC clones are depicted in FIG. 8. Following the insertion of the I-SceI counterselection cassette into intron I of the β-globin gene, the second stage of recombination was carried out by electroporation of a 732 bp ARMS1-LUG2A PCR product carrying the IVS I-5 (G→C) mutation into DH10B (pEBAC/148β::I-SceI/Kan^{R}, pGETrec3.1 ) cells. In view of the above results, the cells were diluted after electroporation in 1ml SOC medium containing cTc and incubated at 37° C. for 1 hour prior to plating on Cm, Amp and cTc. Accurate deletion of the I-SceI/Kan^{R} cassette by recombination is expected to yield the same size PCR product as the unmodified β-globin gene using LUG1A and LUG2A primers. Screening of 93 colonies revealed seven positive clones (7.5%), while in three different experiments the proportion of positive clones ranged from 6-10%, an efficiency much higher than with any other counterselection marker (Nefedov et al, 2000, supra; Nefedov et al, submitted). There were no detectable changes in the 185 kb genomic insert in the recombinant clones after digestion with NotI, or with XhoI (data not shown). High resolution fingerprinting of five clones after EcoRI digestion also showed that there were no detectable differences between the recombinant clones (FIG. 9A, lanes 1-5) and the unmodified pEBAC/148β clone (FIG. 9A, lane C) in the 5.5 kb EcoRI fragment on which the modification took place (arrow), or on any of the other EcoRI fragments. Southern blot analysis with ³²P-labelled LUG probe under low stringency washing also confirmed the presence of all the globin genes (FIG. 9B). Direct DNA sequencing of one of these positive clones with the LUG1A primer confirmed the presence of the IVS I-5 (G→C) mutation in intron I of the β-globin gene, without any other sequence changes in the targeted region (FIG. 10A).

### EXAMPLE 10

### OPTIMISATION OF THE I-SCEI COUNTERSELECTION SYSTEM

The major proportion of colonies surviving cTc selection in the above experiment failed to give a PCR product with the LUG1A/LUG2A primers while over 80% were sensitive to kanamycin, indicating that the I-SceI/Kan^{R} cassette and flanking sequences were deleted. Thus most of the false positive clones appear to result from the insertion of a DSB at the unique I-SceI site, followed by outward degradation and plasmid rescue through recircularisaton as a result of various recombinogenic and repair mechanisms.

A number of factors may contribute to the overall efficiency and specificity of the system for generating correct recombinant clones. Preliminary studies (FIG. 10) indicate that the recE and recT proteins produced during the brief period of induction by L-arabinose prior to electroporation can survive for up to several hours in SOC after electroporation. Thus it may be possible for non-specific recombinant clones to be generated long after the exogenous PCR product has been degraded. To investigate this point, approximately 1 μg of the 447 bp LUG1A-LUG2A PCR product was electroporated into DH10B (pEBAC/148β::I-SceI/Kan^{R}, pGETrec3.1) cells. Following immediate dilution in 1 ml SOC medium, aliquots (250 μl) were collected at 0, 15, 30, and 60 minutes after electroporation. The cells from each time point were quickly cooled, washed three times with LB medium and used for mini-prep DNA extraction. Agarose gel electrophoresis and Southern blot analysis with the PCR product as probe showed recovery of 5-10% of the PCR product at time zero. Most of the PCR product was degraded within 15 minutes, while only a small amount could be detected by 60 minutes after electroporation. Thus specific recombinants may arise mostly within the first 15 minutes after electroporation, while longer incubations in SOC may increase the proportion of false-positive clones.

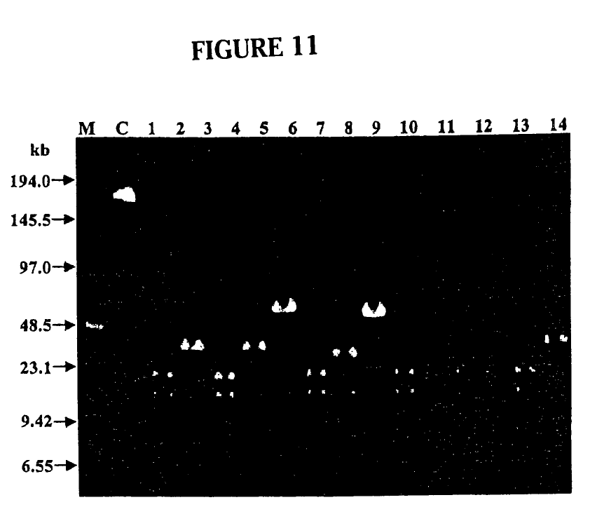

Production of I-SceI is only induced after electroporation, by the inclusion of cTc in the SOC medium. Thus the kinetics of induction of I-SceI after electroporation may be expected to affect the relative efficiency of production of specific to non-specific recombinants. To investigate the production of non-specific recombinant clones DH10B (pEBAC/148β::I-SceI/Kan^{R}, pGETrec3.1) cells were electroporated without any DNA and then diluted in SOC medium containing cTc. Aliquots (250 μl) were collected at 15, 30, 45 and 60 minutes after electroporation and cooled on ice. Mini-prep DNA extraction was carried out and the whole of each sample was digested with I-SceI followed by ethanol precipitation and re-electroporation into normal DH10B cells. Three individual colonies were picked from each time point for miniprep DNA extraction and PFGE analysis after digestion with NotI (FIG. 11). Deletions ranging from about 100 to 160 kb were found from as little as 15 minutes after electroporation, while longer incubations in SOC were associated with an increase in the number of resistant clones and with very large deletions in the genomic insert.

The above studies indicated the possibility of enhancing the proportion of specific recombinant clones by limiting exposure of BAC DNA to various recombinogenic mechanisms after electroporation, while also enhancing counterselection against non-recombinant clones by in vitro digestion with I-SceI. In order to investigate these possibilities, DH10B (pEBAC/148β::I-SceI/Kan^{R}, pGETrec3.1) cells were electroporated with about 1 μg of the 732 bp ARMS1-LUG2A PCR product from normal genomic DNA. The reaction was immediately diluted with 1 ml SOC medium supplemented with cTc and incubated at 37° C. Aliquots (300 μl) were collected at 15, 30, and 60 minutes after electroporation. DNA was extracted from each time point, digested with I-SceI and re-electroporated into normal DH10B cells. 50-100 clones were obtained from each electroporation under these conditions. PCR analysis of 30-50 clones from each time point indicated accurate removal of the counterselection cassette in 11% of the clones after 15 minutes, rising to 33% at 30 minutes and 31% at 60 minutes. Most of the remaining clones were sensitive to kanamycin, indicating the deletion of the counterselection cassette with variable amounts of flanking sequences.

### EXAMPLE 11

### INTRODUCTION OF THE IVS II-654 (C→T) MUTATION THROUGH AN IN VITRO COUNTERSELECTION SYSTEM

The usefulness of the in vitro counterselection protocol for the insertion of the IVS 11-654 mutation into the β-globin gene was investigated. The same pEBAC/148β::I-SceI/Kan^{R} construct was used in this experiment as for the IVS I-5 mutation, although the IVS II-654 mutation is about 900 bp downstream from the site of insertion of the counterselection cassette. In brief, a 1708 bp PCR product obtained with the ARMS1-HbbRev primers from the genomic DNA of a patient homozygous for the IVS II-654 mutation was electroporated into E. coli DH10B (pEBAC/148β::I-SceI/Kan^{R}, pGETrec3.1) cells. Cells were incubated in 1 ml SOC containing cTc at 37° C. for 30 minutes prior to mini-prep DNA extraction and in vitro digestion with I-SceI. The BAC DNA was ethanol precipitated and re-electroporated into DH10B electrocompetent cells. Although this process may result in the loss of some recombinant molecules, PCR screening with LUG1A/LUG2A primers of 50 colonies revealed 15 clones (30%) positive for the 447 bp product, indicating correct excision of the counterselection cassette. Restriction analysis of five individual clones did not show any unwanted rearrangements (data not shown), while sequence analysis of one of these clones confirmed the presence of the IVS II-654 (C→T) mutation in intron II of the β-globin gene, without any other changes in the targeted region (FIG. 10B).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Antoch, M. P., et al., (1997) Cell 89:655-667.

Arakawa, Y., Wacharotayankun, R., Ohta, M, Shoji, K., Watahiki, M., Horri, T. and Kato, N. (1991) Gene 104:81-84.

Bej, A. K., Perlin, M. H and Atlas, R. M. (1988) Appl. Environ. Microbiol. 54:2472-2477.

Bernard, P. (1996) Biotechniques 21.320-323.

Betlach, M., Hershfield, V., Chow, L., Brown, W., Goodman, H. and Boyer, H. W. (1976) Fed. Proc. 35:2037-2043.

Brinster, R. L. et al. (1988) Proc. Natl. Acad. Sci. USA 85:836-840.

Chan, R. Y., Palfree, R. G., congote, L. F. and Solomon, S. (1994) DNA Cell Biol. 13:311-319.

Choi, T., Huang, M., Gorman, C. and Jaenisch, R. (1991) Mol. Cell. Biol. 11:3070-3074.

Harada, K., Nishizaki, T., Maekawa, K., Kubota, H., Suzuki, M., Ohno, T., Saski, K. and Soeda, E. (2000) Genomics 67:268-272.

Hejna, J. A. et al., (1998) Nucleic Acids Res. 26:1124-1125.

Hubert, R. S. el al. (1997) Genomics 41:218-226.

Ioannou, P. A., Amemiya, C. T., Garnes, J., Kroisel, P. M., Shizuya. H., Chen, C., Batzer, M. A. and de Jong, P. J. (1994) Nat. Genet. 6:84-89.

Jaenisch, R. (1988) Science 240:1468-1474.

Kuhn, I., Stephenson, F. H., Boyer, H. W. and Greene, P. J. (1986) Gene 42:253-263.

Laemmli, U. K. (1970) Nature 227:680-685.

Lamb, B. T. et al. (1993) Nat. Genet. 5:22-30.

Marshall, E. and Pennisi, E. (1998) Science 280:994-995.

Narayanan, K., Williamson, R., Zhang, Y., Stewart, A. F. and Ioannou, P. A. (1999) Gene Ther. 6:442-447.

Nechiporuk, T. et al. (1997) Genomics 44:321-329.

Nefedov, M., Williamson, R. and Ioannou, P. A. (2000) Nucleic Acids Res. 28:e79.

Orford, M., Nefedov, M., Vadolas, J., Zaibak, F. Williamson, R. and Ioannous, P. A. (2000) Nudleic Acids Res. 28:e84.

Osoegawa, K., de Jong, P. J., Frengen, E. Ioannou, P. A. (1999) Construction of Bacterial Atificial Chromosome (BAC/PAC) Libraries, in Current Protocols in Human Genetics, Unit 5.15 Eds Dacopoli, N. C., Haines, J. L., Korf, B. R., Moir, D. T., Morton, C. C., Seidman, C. E., Seidman, J. G., Smith, D. R. John Wiley & Sons, New York.

Peterson, K. R., et al. (1996) Proc. Natl. Acad. Sci. USA 93:6605-6609.

Pierce, J. C., Sauer, B. and Sternberg, N. (1992) Proc. Natl. Acad. Sci. 89:2056-2060.

Porcu, S., et al. (1997) Blood 90:4602-4609.

Posfai, G. et al. (1999) Nucleic Acids Res. 27:4409-4415.

Raguz, S., et al. (1998) Dev. Biol. 201:26-42.

Reid, L. H., Davies, C., Cooper, P. R. Crider-Miller, S. J., Sait, S. N., Nowak, N. J., Evans, G., Stanbridge, E. J., de Jong, P., Shows, T. B., Weissman, b. e. and Higins, M. J. (1997) Genomics 43:366-375.

Sambrook, J., Fritsch, E. F. and Maniatis, T. (1989) Molecular cloning: A laboratory manual, 2^{nd} ed. Cold Spring Harbor Lab. Press. Cold Spring Harbor.

Shizuya, H., Birren, B., Kim, U. J., Mancino, V., Slepak, T., Tachiiri, Y. and Simon, M. (1992) Proc. Natl. Acad. Sci. 89:8794-8797.

Sukumaran, P. K., Nakatsuji, T. Gardiner, M. B., Reese, A. L., Gilman, J. G. and Huisman, T. H. Nucleic Acids Res. 11:4635-4643.

Venter, J. C. et al. (1998) Science 280:1540-1542.

Venter, J. C., Smith, H. L. and Hood, L. (1996) Nature 381:364-366.

Wooster, R. et al. (1995) Nature 378:789-792.

1. A method for selecting a modified nucleic acid molecule or derivative or analogue thereof, said method comprising the steps of facilitating the interaction, in a host cell, of a counterselection marker with an unmodified nucleic acid molecule at a target modification region, which counterselection marker can facilitate the inducible degradation of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the degradation activity of said counterselection marker.

2. A method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said endonuclease.

3. A method for selecting a modified nucleic acid molecule or derivative or analogue thereof said method comprising the steps of facilitating the interaction, in a host cell, of a counterselection marker with an unmodified nucleic acid molecule at a target modification region, which counterselection marker can facilitate the inducible restriction endonuclease-mediated cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing the cleavage activity of said restriction endonuclease.

4. A method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence encoding a restriction endonuclease or derivative, homologue, equivalent or mimetic of said restriction endonuclease and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing expression of said restriction endonuclease.

5. The method according to claim 4 wherein said counterselection marker is a nucleic acid sequence encoding EcoRI or derivative, homologue, equivalent or mimetic thereof and said selection step comprises inducing expression of said EcoRI.

6. A method for selecting a modified nucleic acid molecule or derivative or analogue thereof said method comprising the steps of facilitating the interaction, in a host cell, of a counterselection marker with an unmodified nucleic acid molecule at a target modification region, which counterselection marker is a nucleic acid sequence incorporating a restriction endonuclease cleavage site and which can facilitate the inducible cleavage of said nucleic acid molecule, facilitating the modification of said nucleic acid molecule at said target modification region, which modification comprises the functional deletion of said counterselection marker from said nucleic acid molecule and selecting said modified nucleic acid molecule wherein said selection step comprises inducing cleavage at said restriction endonuclease cleavage site.

7. The method according to claim 6 wherein said counterselection marker is a nucleic acid sequence incorporating an I-SceI cleavage site and said selection step comprises inducing cleavage at said I-SceI cleavage site.

8. The method according to any one of claims 4-7 wherein said modified nucleic acid molecule is a modified BAC or derivative or analogue thereof and said host cell is a DH10B cell.

9. The method according to any one of claims 4-7 wherein said modified nucleic acid molecule is a modified PAC or derivative or analogue thereof and said host cell is a DH10B cell.

10. A modified nucleic acid molecule selected in accordance with the method of any one of claims 1-4 or 6.

11. The modified nucleic acid molecule according to claim 10 wherein said modified nucleic acid molecule is a modified BAC or a modified PAC.

12. A method for the therapeutic and/or prophylactic treatment of a subject, said method comprising administering an effective amount of a nucleic acid molecule, which nucleic acid molecule has been modified in accordance with the method of any one of claims 1-4 or 6.

13. A method of screening, said method comprising utilising an effective amount of a nucleic acid molecule, which nucleic acid molecule has been modified in accordance with the method of any one of claims 1-4 or 6.

14. The method according to claim 13, wherein said screening method is diagnostic screening.

15. A pharmaceutical composition comprising nucleic acid molecules modified in accordance with any one of claims 1-4 or 6 together with one or more pharmaceutically acceptable carriers and/or diluents.

16. A kit for facilitating selection of a modified nucleic acid molecule in accordance with the method of any one of claims 1-4 or 6, said kit comprising compartments adapted to contain any one or more of a counterselection marker, reagents useful for facilitating modification of a nucleic acid molecule and reagents useful for facilitating selection of said modified nucleic acid molecule.
